(19) United States
(12) Patent Application Publication
(10) Pub. No.: US 2010/0104577 A1
(43) Pub. Date: Apr. 29, 2010

(54) METHODS AND MATERIALS RELATED TO ANTI-A (BETA) ANTIBODIES

(76) Inventors: Todd E. Golde, Ponte Vedra Beach, FL (US); Pritam Das, Ponte Vedra Beach, FL (US); Karen R. Jansen-West, Jacksonville Beach, FL (US); Yona R. Levites, Jacksonville, FL (US)

Correspondence Address: FISH & RICHARDSON P.C., PO BOX 1022, MINNEAPOLIS, MN, 55440-1022, US

(21) Appl. No.: 12/444,984

(22) PCT Filed: Oct. 10, 2007
(86) PCT No.: PCT/US07/80988

§ 371 Date: Apr. 9, 2009

Related U.S. Application Data
(60) Provisional application No. 60/850,919, filed on Oct. 10, 2006.

(51) Int. Cl. A61K 39/395; C12N 15/86; C07H 21/04; A61K 48/00; C07K 16/32

(52) U.S. Cl. 424/155.1; 424/93.2; 530/388.5; 435/456; 536/23.2

(57) ABSTRACT

This document provides methods and materials related to anti-Aβ antibodies. For example, anti-Aβ antibodies, methods for making anti-Aβ antibodies, and methods for using an anti-Aβ antibody to treat or prevent an amyloid condition (e.g., AD).

### CROSS REFERENCE RELATED APPLICATIONS

This document claims priority to U.S. Provisional Application Ser. No. 60/850,919, filed on Oct. 10, 2006, the contents of which are herein incorporated by reference in their entirety.

### STATEMENT AS TO FEDERALLY SPONSORED RESEARCH

Funding for the work described herein was provided by the federal government under grant number AG 021875 awarded by the National Institute of Health. The federal government has certain rights in the invention.

### BACKGROUND

1. Technical Field

This document provides methods and materials related to anti-Aβ antibodies and treating amyloid conditions (e.g., Alzheimer's disease).

2. Background Information

It is hypothesized that the process that results in accumulation of Aβ as amyloid triggers the complex pathological changes that ultimately lead to cognitive dysfunction in Alzheimer's disease (AD). However, there is substantial debate as to the form or forms of Aβ aggregates that damage the brain. Aβ accumulates as amyloid in senile plaques and cerebral vessels, but it is also found in diffuse plaques recognized by antibodies but not classic amyloid stains. Although a minor component of the Aβ species produced by processing of amyloid precursor protein (APP), the highly amyloidogenic 42 amino acid form of Aβ (Aβ1-42) and amino terminally truncated forms of Aβ1−42 (Aβ1x−42) are the predominant species of Aβ typically found in both diffuse and senile plaques within the AD brain. However many other forms of Aβ (e.g., Aβ1-40 or Aβ1x−40) are also present, especially in cerebrovascular amyloid deposits. Additionally, soluble Aβ aggregates referred to as oligomers, which in rodents can acutely disrupt neuronal function, appear to accumulate in the AD brain. The exact composition and levels of these oligomers in the brain parenchyma has yet to be elucidated.

### SUMMARY

This document provides methods and materials related to anti-Aβ antibodies. For example, this document provides anti-Aβ antibodies, methods for making anti-Aβ antibodies, and methods for using an anti-Aβ antibody to inhibit amyloid plaques.

In general, one aspect of this document features a substantially pure antibody having binding affinity for an Aβ epitope, wherein the Aβ epitope is the epitope of scFv40.1, scFv42.2, or scFv9. The antibody has less than 10⁴ mol⁻¹ binding affinity for Aβ1-38. The antibody can have less than two percent cross reactivity with Aβ1-38. The antibody can be monoclonal. The antibody can comprise the sequence set forth in SEQ ID NO:2. The antibody can comprise the sequence set forth in SEQ ID NO:3. The antibody can be an scFv40.1 antibody. The antibody can be an scFv42.2 antibody.

In another aspect, this document features a method for inhibiting Aβ plaque formation in a mammal. The method comprising administering an antibody to the mammal, wherein the antibody has binding affinity for an Aβ epitope, wherein the Aβ epitope is the epitope of scFv40.1, scFv42.2, or scFv9.

In another aspect, this document features a nucleic acid construct comprising a nucleic acid sequence encoding the amino acid sequence set forth in SEQ ID NO:2, 3, or 4. The construct can be an AAV vector.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Specificity of mAbs Ab40.1 and Ab42.2. A. Serial dilutions of Aβ40, Aβ42 and Aβ38 were used to determine the cross reactivity of mAb Ab42.2 by capture ELISA. mAb Ab42.2 was used as capture and Ab9-HRP as detection. B. Serial dilutions of Aβ40, Aβ42 and Aβ38 were used to determine the cross reactivity of mAb Ab40.1 by capture ELISA. mAb Ab9 was used as capture and Ab40.1 was used as detection. C. Schematic depicting the method for capture ELISA of Aβ/biotinylated mAb complex in plasma. D. Specificity of mAbs Ab42.2 and Ab40.1 in vivo. 500 μg of biotinylated mAbs Ab42.2, Ab40.1 and Ab9 were injected i.p. into TgBRI-Aβ40 and TgBRI-Aβ42 transgenic mice (n=3/group). 72 hours after the injection, levels of Aβ-antibody complexes in plasma were determined using capture ELISA as illustrated in C. Plasma levels of Aβ40 are 1000 μM in TgBRI-Aβ40 and Aβ42 levels are 1000 μM in TgBRI-Aβ42 mice, respectively. No Aβ42 can be detected in the plasma of TgBRI-Aβ40 mice, and no Aβ40 can be detected in the plasma of TgBRI-Aβ42 mice.

FIG. 2. Effect of immunization with C-terminal specific antibodies on Aβ levels in brains of Tg2576 mice. A and B. 7-month-old Tg2576 mice (n=6/group) were immunized with 500 μg of Ab40.1 and Ab42.2 mAbs, biweekly, for 4 months and compared to immunization with mAb Ab9. Control mice received PBS. Mice were killed following treatment and both SDS soluble (SDS Aβ) and SDS-insoluble formic-acid extracted (FA Aβ) fractions analyzed by capture ELISA. SDS and FA Aβ1-40 levels in control mice were 123±27 and 3613±610 pmol/g, respectively; SDS and FA Aβ1-42 levels in control mice were 44±4 and 840±180 pmol/g, respectively. A. SDS-soluble Aβ1-42 and Aβ1-40 levels *p<0.05, **p<0.01 vs control. B. SDS-insoluble FA-soluble Aβ42 and Aβ40 levels. *p<0.05, **p<0.01 vs control. C. Representative immunostained sections for amyloid plaques from brains of mAb immunized 7-month-old Tg2576 mice. Magnification 100×. D. Quantitative image analysis of amyloid plaque burden in the neocortex of immunized 7-month-old Tg2576 mice. *p<0.001 vs control. 11-month-old Tg2576 mice (n=6/group) were immunized with 500 μg of Ab40.1 and Ab42.2 mAbs, biweekly, for 4 months and compared to immunization with mAb Ab9. E. SDS-soluble Aβ1-42 and Aβ1-40 levels. B. SDS-insoluble FA-soluble Aβ42 and Aβ40 levels.

FIG. 3. Effect of immunization with anti-Aβ antibodies on Aβ levels in brains of CRND8 mice. A and B. 3-month-old CRND8 mice (n=6/group) were immunized with 500 μg of Ab9 or Ab42.2 mAbs, weekly, for 8 weeks. Control mice received PBS. Mice were killed following treatment and both SDS soluble (SDS Aβ) and SDS-insoluble formic-acid extracted (FA Aβ) fractions analyzed by capture ELISA. SDS and FA Aβ1-40 levels in control mice were 217±40 and 563±95 pmol/g, respectively; SDS and FA Aβ1-42 levels in control mice were 189±12 and 636±51 pmol/g, respectively. A. SDS-soluble Aβ1-42 and Aβ1-40 levels *p<0.05, **p<0.01 vs control. B. SDS-insoluble FA-soluble Aβ42 and Aβ40 levels. C. Representative immunostained sections for amyloid plaques from brains of mAb immunized CRND8 mice. Magnification 40×. D. Quantitative image analysis of amyloid plaque burden in the neocortex of immunized CRND8 mice. *p<0.05, **p<0.01 vs control.

FIG. 4. Effect of direct cortical injections with anti-Aβ mAbs on Aβ plaque burdens in 18-month-old Tg2576 mice. Mice were injected in the frontal cortex with 1 μg each the following antibodies: control mouse IgG, Ab9, Ab5, Ab3, Ab2, Ab40.1 and Ab42.2. A. Representative pictures of immunostained Aβ plaques taken from injection sites in cortex following injection with mAbs Ab9, control IgG (Equitech-Bio, Inc.), Ab42.2 and Ab40.1. C. Quantitative analysis of immunostained amyloid plaque burdens in mice following mAbs injections. *p<0.01 vs mouse IgG. B. Representative pictures of Thio-S positive Aβ plaques taken from injection sites in cortex following injection with mAbs Ab9, control IgG, Ab42.2 and Ab40.1. Magnification 100×D. Quantitative analysis of Thio-S positive amyloid plaque burdens in mice following mAbs injections.

FIG. 5. Effect of immunization with N-terminal specific antibodies on Aβ levels in brains of 10-month-old Tg2576 mice. A. Unfixed frozen cryostat serial sections of the human AD tissue (hippocampus) were stained with Ab9, Ab3, Ab5, Ab2, Ab40.1 and Ab42.2 antibodies. Representative plaque staining is shown. Magnification 400×. B. Quantitative image analysis of the average fluorescent intensity level per plaque following mAb binding. *p<0.001 vs Ab40.1, †p<0.05 vs Ab2. C and D. Aβ levels in brains of Ab2, Ab5, Ab9 and Ab3 immunized Tg2576 mice. 10-month-old Tg2576 mice (n=6/group) were immunized with 500 μg/biweekly with N-terminal mAbs for 4 months. Mice were sacrificed following treatment and brain tissue was subject to a two-step SDS/Formic Acid extraction. Both SDS-Aβ (C) and SDS-insoluble FA-Aβ (D) were analyzed by capture ELISA. SDS and FA Aβ1-40 levels in control mice were 1115±72 and 4675±430 pmol/g, respectively; SDS and FA Aβ1-42 levels in control mice were 348±54 and 737±62 pmol/g, respectively. *p<0.05, **p<0.01 vs control.

FIG. 6. Aβ and mAb levels following passive immunization with mAb9. A. 3-month-old Tg2576 mice were dosed i.p. with 500 μg (1600 pmol) biotinylated mAb9. Aβ levels were measured at different time points by ELISA with end-specific anti-Aβ40 mAb (Ab40.1) as capture and 4G8-HRP as detection. B. Levels of Aβ bound by biotinylated mAb9 in the plasma were measured at different time points by ELISA using mAb40.1 as capture and Neutravidin-HRP as detection. n=4 per group, *p<0.001 vs control. C. Plasma from Tg2576 mice dosed with biotinylated mAb9 or biotinylated mouse IgG was fractionated by size-exclusion chromatography. Levels of Aβ in each fraction were measured by ELISA. D. Aβ:biotinylated mAb complex in the plasma of treated Tg2576 mice was immunoprecipitated with streptavidin beads, dissolved in SDS-PAGE sample buffer and subjected to a 12% Bis-Tris electrophoresis gel. Aβ was detected by mAb9 (1:1000). E and F. 3-month-old non-transgenic mice were dosed i.p with 500 μg (1600 pmol) biotinylated Ab9 (E) or with complex of ˜1600 pmol biotinylated mAb9 and 3200 pmol Aβ40 (F). E. Biotinylated mAb9 levels in the plasma were measured by direct ELISA (see methods). F. Levels of Aβ bound by biotinylated mAb in the plasma were measured at different time points by ELISA. n=4 per group, *p<0.01, **p<0.001 vs control.

FIG. 7. Effects of mAb9 on Aβ levels in the brains of Tg2576 mice. 3-month-old Tg2576 mice were dosed with 500 μg biotinylated mAb9. Six hours-14 days later, mice were perfused with PBS, and Aβ levels in GuHCl (A), TBS (B) and RIPA (C and D) brain extracts were detected by ELISA using end-specific anti-Aβ40 mAb40.1 or anti-Aβ42 mAb42.2 as capture and 4G8-HRP as detection. n=4 per group. (E) 3-month-old Tg2576 mice were dosed with 500 μg biotinylated mAb9 every week for 4 weeks. Mice were killed 24 hours after the final mAb administration. Aβ levels in RIPA brain extracts were detected by ELISA. n=4 per group.

FIG. 8. Effects of mAb9 on Aβ levels in the plasma and brains of BRI-Aβ42B mice. 3-month-old BRI-Aβ42B mice were dosed with 500 μg biotinylated mAb9. Six or 24 hours later mice were bled and perfused with PBS. Aβ levels in the plasma (A) as well as in GuHCl (B), TBS (C) and RIPA (D) brain extracts were detected by ELISA using end-specific anti-Aβ42 mAb42.2 as capture and 4G8 mAb as detection. n=4 per group.

FIG. 9. Effects of mAb9 on Aβ levels in the CSF. 3-month-old Tg2576 mice were dosed with 500 μg (1600 pmol) biotinylated Ab9, i.p. A. Total levels of Aβ40 and Aβ42 in the CSF were measured using Ab40.1 or Ab42.2 as capture and 4G8 as detection. B. Levels of Aβ40 bound by mAb in the CSF were measured after 6 or 24 hours by capture ELISA using Ab40.1 as capture and Neutravidin-HRP as detection. n=4, *p<0.01, **p<0.001 vs control. Data is shown from a single experiment. Similar data were seen in two other independent studies. C. 3-month-old Tg2576 mice were injected with 50 μg (160 pmol) biotinylated mAb9 bound to ˜320 pmol Aβ, ICV. The levels of Aβ bound by mAb in the plasma and CSF were measured by capture ELISA. n=2.

FIG. 10. Effects of three anti-Aβ antibodies mAb3, mAb42.2 and mAb 40.1 on Aβ levels on Aβ levels in plasma, brains and CSF of Tg2576 mice. 3-month-old Tg2576 mice were dosed with 500 μg biotinylated mAb3, mAb42.2 and mAb 40.1. Six or 24 hours later plasma and CSF were extracted and mice were subsequently perfused with PBS. For mAb3, Aβ40 and Aβ42 levels in the plasma (A), RIPA brain extracts (B) and CSF (C) were detected by ELISA using mAb42.2 (Ab42) or mAb40.1 (Ab40) as capture and 4G8 mAb as detection. To avoid possible interference only total Aβ levels were measured in plasma, brain and CSF of mAb40.1 and mAb 42.2 treated mice using ELISA with mAb9 as capture and 4G8 mAb as detection (D, E and F), n=4 per group, *p<0.05, **p<0.01 vs control.

FIG. 11. Expression and binding properties of anti-Aβ scFvs. 293T HEK cells were transiently transfected with scFv9, scFv40.1 and scFv42.2 in pSecTag. A. Sequence alignment of anti-Aβ scFvs: scFv ns (SEQ ID NO:1), scFv40.1 (SEQ ID NO:2), scFv42.2 (SEQ ID NO:3), and scFv9 (SEQ ID NO:4). B. Western blot of a 1% Triton lysate and conditioned media, detected with anti-His primary antibody and anti-rabbit-HRP secondary antibody showing expression of the anti-Aβ scFvs. C. Western blot of a pull-down of conditioned media with fAβ, detected with anti-His primary antibody and anti-rabbit-HRP secondary antibody showing that the anti-Aβ scFvs maintain the binding selectivity of the parent antibodies. D. Conditioned media from scFv9, scFv40.1 and scFv42.2 transfected cells was tested in an ELISA with Aβ40 or Aβ42 as capture and anti-myc-HRP as detection. *p<0.01 vs control. E. Paraffin sections of Tg2576 mice brains were stained with conditioned media from scFv transfected cells (bottom panel) and anti-His primary antibody or with a corresponding parent anti-Aβ mAb (top panel). Representative plaque staining is shown. Magnification 200×.

FIG. 12. Expression of an anti-Aβ scFvs in the neonatal mouse brain using AAV1. A. P0 Swiss Webster pups were injected ICV with AAV1-hGFP, total of 4×10¹² genomes. AAV expression in mouse brain 3 weeks and 10 months post injection. Magnification 40× (top panel) and 200× (bottom panel). B. Newborn CRND8 mice were injected ICV with AAV1 scFv. After 3 weeks, brain paraffin sections were analyzed for scFv expression using anti-His primary antibody and anti-rabbit secondary antibody. Magnification 200×.

FIG. 13. Anti-Aβ scFvs attenuate Aβ deposition in 5 month old CRND8 mice. Newborn CRND8 mice were injected ICV with AAV1 scFv9 and scFv42.2. Control mice received AAV1-hGFP. Five months later, mice were sacrificed following treatment and one hemibrain processed for immunohistochemistry and the other for biochemical analysis. A. Representative immunostained sections for amyloid plaques from brains of scFv treated CRND8 mice. Magnification 40×. B. Aβ levels in the SDS-soluble and SDS-insoluble FA-soluble fractions analyzed by Aβ sandwich ELISA. n=5. *p<0.05 vs control.

FIG. 14. Anti-Aβ scFvs attenuate Aβ deposition in 3 month old CRND8 mice. Newborn CRND8 mice were injected ICV with AAV1 expressing scFv9, scFv40.1 and scFv42.2. Control mice received AAV1scFv ns or PBS. Three months later mice were sacrificed following treatment. One hemibrain was used for immunohistochemistry and the other for biochemical analysis. A. Representative immunostained sections for amyloid plaques from brains of scFv treated CRND8 mice. Magnification 40×. B.

Quantitative image analysis of amyloid plaque burden in the neocortex of scFv treated CRND8 mice. *p<0.05 vs control. C. Aβ levels in the SDS-soluble. D. An Aβ/scFv complex in plasma was detected by ELISA with a capture antibody specific to the free end of Aβ (for scFv9 mAb40.1, for scFv40.1 and scFv42.2 mAb9) and anti-myc-HRP as detection. n=7, *p<0.05 vs non-specific scFv, **p<0.01 vs non-specific scFv, ***p<0.005 vs non-specific scFv.

FIG. 15 is a listing of nucleic acid sequences that encode the amino acid sequence set forth in SEQ ID NOs:1-4.

FIG. 16. Anti-amyloid scFvs. A) Schematic of modified amyloid pulldown “panning” method for identifying anti-amyloid scFvs. B) Representative ELISA reactivity of putative anti-amyloid scFv phagemids against three amyloids (fAb42, hA AVS41 and hA CS35. Anti-ubiquitin scFv phagemid is used as a control. C) A table of the scFvs successfully expressed in 293 cells. The sequence of pulldowns used to pan for these scFvs and the “randomized” sequences of the VH and VL regions are shown. D) Representative amyloid pulldown experiment using conditioned media from stable 293 cells expressing anti-Aβ (scFv9, scFv42.2) and anti-amyloid scFvs (scFv21,scFv82). Aβ amyloid or hA from AVS41, CS35, or BOC polypeptides were used to assess reactivity to amyloid. Ni refers to nickel affinity agarose bead pulldown as a positive control for scFv in the conditioned media. Strept refers to streptavidn agarose bead pulldown used as a control for non-specific binding. E) ELISA reactivity of anti-Aβ, anti-BSA and anti-amyloid scFvs (21,82 B8) against plates coated 1 μg/mL monomeric Aβ, SDS oligomer and Aβ amyloid fibrils. F) Multiple anti-amyloid scFv reduce Aβ levels in CRND8 mice. Preliminary studies of rAAV1 delivered anti-amyloid scFvs shows that multiple anti-amyloid scFvs appear to reduce biochemical Aβ loads.

FIG. 17 is a listing of nucleic acid and amino acid sequences of the indicated antibodies.

### DETAILED DESCRIPTION

This document provides methods and materials related to anti-Aβ antibodies. For example, this document provides anti-Aβ antibodies, methods for making anti-Aβ antibodies, and methods for using an anti-Aβ antibody to treat or prevent an amyloid condition (e.g., AD). In some cases, the antibodies provided herein can bind to Aβ1-40 or Aβ1-42 with little or no detectable binding to other Aβ peptides. For example, an antibody provided herein can bind to human Aβ1-40 without binding to human Aβ1-38. In some cases, the antibodies provided herein can bind to Aβ1-40 with little or no detectable binding to Aβ1-38 or Aβ1-42. For example, an antibody provided herein can bind to human Aβ1-40 without binding to human Aβ1-38 or Aβ1-42. An example of an antibody having the ability to bind to Aβ1-40 with little or no detectable binding to Aβ1-38 or Aβ1-42 includes, without limitation, mAb40.1. In some cases, the antibodies provided herein can bind to Aβ1-42 with little or no detectable binding to Aβ1-38 or Aβ1-40. For example, an antibody provided herein can bind to human Aβ1-42 without binding to human Aβ1-38 or Aβ1-40. An example of an antibody having the ability to bind to Aβ1-42 with little or no detectable binding to Aβ1-38 or Aβ1-40 includes, without limitation, mAb42.2.

The term “antibody” as used herein refers to intact antibodies as well as antibody fragments that retain some ability to bind an epitope. Such fragments include, without limitation, Fab, F(ab′)2, and Fv antibody fragments. The term “epitope” refers to an antigenic determinant on an antigen to which the paratope of an antibody binds. Epitopic determinants usually consist of chemically active surface groupings of molecules (e.g., amino acid or sugar residues) and usually have specific three dimensional structural characteristics as well as specific charge characteristics.

The antibodies provided herein can be any monoclonal or polyclonal antibody having specific binding affinity for an Aβ polypeptide (e.g., an Aβ1-40 or Aβ1-42 polypeptide) with little or no detectable binding to Aβ1-38. Such antibodies can be used in immunoassays in liquid phase or bound to a solid phase. For example, the antibodies provided herein can be used in competitive and non competitive immunoassays in either a direct or indirect format. Examples of such immunoassays include the radioimmunoassay (RIA) and the sandwich (immunometric) assay. In some cases, the antibodies provided herein can be used to treat or prevent amyloid conditions (e.g., AD). For example, an antibody provided herein can be conjugated to a membrane transport sequence to form a conjugate that can be administered to cells in vitro or in vivo. Examples of membrane transport sequences include, without limitation, AALALPAVLLALLAP (Rojas et al., J Biol Chem, 271(44):27456-61 (1996)) and KGEGAAVLLPVLLAAPG (Zhao et al., Apoptosis, 8(6):631-7 (2003) and Zhao et al., Drug Discov Today, 10(18):1231-6, (2005)). Nucleic acids encoding these membrane transport sequences can be readily designed by those of ordinary skill in the art.

Antibodies provided herein can be prepared using any method. For example, any substantially pure Aβ polypeptide, or fragment thereof, can be used as an immunogen to elicit an immune response in an animal such that specific antibodies are produced. Thus, Aβ1-40 or Aβ1-42 or fragments containing small polypeptides can be used as an immunizing antigen. In addition, the immunogen used to immunize an animal can be chemically synthesized or derived from translated cDNA. Further, the immunogen can be conjugated to a carrier polypeptide, if desired. Commonly used carriers that are chemically coupled to an immunizing polypeptide include, without limitation, keyhole limpet hemocyanin (KLH), thyroglobulin, bovine serum albumin (BSA), and tetanus toxoid.

The preparation of polyclonal antibodies is well-known to those skilled in the art.

See, e.g., Green et al., Production of Polyclonal Antisera, in IMMUNOCHEMICAL PROTOCOLS (Manson, ed.), pages 15 (Humana Press 1992) and Coligan et al., Production of Polyclonal Antisera in Rabbits, Rats, Mice and Hamsters, in CURRENT PROTOCOLS IN IMMUNOLOGY, section 2.4.1 (1992). In addition, those of skill in the art will know of various techniques common in the immunology arts for purification and concentration of polyclonal antibodies, as well as monoclonal antibodies (Coligan, et al., Unit 9, Current Protocols in Immunology, Wiley Interscience, 1994).

The preparation of monoclonal antibodies also is well-known to those skilled in the art. See, e.g., Kohler & Milstein, Nature 256:495 (1975); Coligan et al., sections 2.5.1 2.6.7; and Harlow et al., ANTIBODIES: A LABORATORY MANUAL, page 726 (Cold Spring Harbor Pub. 1988). Briefly, monoclonal antibodies can be obtained by injecting mice with a composition comprising an antigen, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well established techniques. Such isolation techniques include affinity chromatography with Protein A Sepharose, size exclusion chromatography, and ion exchange chromatography. See, e.g., Coligan et al., sections 2.7.1 2.7.12 and sections 2.9.1 2.9.3; Barnes et al., Purification of Immunoglobulin G (IgG), in METHODS IN MOLECULAR BIOLOGY, VOL. 10, pages 79 104 (Humana Press 1992).

In addition, methods of in vitro and in vivo multiplication of monoclonal antibodies is well known to those skilled in the art. Multiplication in vitro can be carried out in suitable culture media such as Dulbecco's Modified Eagle Medium or RPMI 1640 medium, optionally replenished by mammalian serum such as fetal calf serum, or trace elements and growth sustaining supplements such as normal mouse peritoneal exudate cells, spleen cells, and bone marrow macrophages. Production in vitro provides relatively pure antibody preparations and allows scale up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells (e.g., osyngeneic mice) to cause growth of antibody producing tumors. Optionally, the animals are primed with a hydrocarbon, especially oils such as pristane (tetramethylpentadecane) prior to injection. After one to three weeks, the desired monoclonal antibody is recovered from the body fluid of the animal.

In some cases, the antibodies provided herein can be made using non-human primates. General techniques for raising therapeutically useful antibodies in baboons can be found, for example, in Goldenberg et al., International Patent Publication WO 91/11465 (1991) and Losman et al., Int. J. Cancer, 46:310 (1990).

In some cases, the antibodies can be humanized monoclonal antibodies. Humanized monoclonal antibodies can be produced by transferring mouse complementarity determining regions (CDRs) from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions when treating humans. General techniques for cloning murine immunoglobulin variable domains are described, for example, by Orlandi et al., Proc. Nat'l. Acad. Sci. USA, 86:3833 (1989). Techniques for producing humanized monoclonal antibodies are described, for example, by Jones et al., Nature, 321:522 (1986); Riechmann et al., Nature, 332:323 (1988); Verhoeyen et al., Science, 239:1534 (1988); Carter et al., Proc. Nat'l. Acad. Sci. USA, 89:4285 (1992); Sandhu, Crit. Rev. Biotech., 12:437 (1992); and Singer et al., J. Immunol., 150:2844 (1993).

Antibodies provided herein can be derived from human antibody fragments isolated from a combinatorial immunoglobulin library. See, for example, Barbas et al., METHODS: A COMPANION TO METHODS IN ENZYMOLOGY, VOL. 2, page 119 (1991) and Winter et al., Ann. Rev. Immunol., 12: 433 (1994). Cloning and expression vectors that are useful for producing a human immunoglobulin phage library can be obtained, for example, from STRATAGENE Cloning Systems (La Jolla, Calif.). In addition, antibodies provided herein can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens and can be used to produce human antibody secreting hybridomas. Methods for obtaining human antibodies from transgenic mice are described by Green et al., Nature Genet., 7:13 (1994); Lonberg et al., Nature, 368:856 (1994); and Taylor et al., Int. Immunol., 6:579 (1994).

Antibody fragments can be prepared by proteolytic hydrolysis of an intact antibody or by the expression of a nucleic acid encoding the fragment. Antibody fragments can be obtained by pepsin or papain digestion of intact antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. In some cases, an enzymatic cleavage using pepsin can be used to produce two monovalent Fab′ fragments and an Fc fragment directly. These methods are described, for example, by Goldenberg (U.S. Pat. Nos. 4,036,945 and 4,331,647). See, also, Nisonhoff et al., Arch. Biochem. Biophys., 89:230 (1960); Porter, Biochem. J., 73:119 (1959); Edelman et al., METHODS IN ENZYMOLOGY, VOL. 1, page 422 (Academic Press 1967); and Coligan et al. at sections 2.8.1 2.8.10 and 2.10.1 2.10.4.

Other methods of cleaving antibodies, such as separation of heavy chains to form monovalent light heavy chain fragments, further cleavage of fragments, or other enzymatic, chemical, or genetic techniques may also be used provided the fragments retain some ability to bind (e.g., selectively bind) its epitope.

The antibodies provided herein can be substantially pure. The term “substantially pure” as used herein with reference to an antibody means the antibody is substantially free of other polypeptides, lipids, carbohydrates, and nucleic acid with which it is naturally associated in nature. Thus, a substantially pure antibody is any antibody that is removed from its natural environment and is at least 60 percent pure. A substantially pure antibody can be at least about 65, 70, 75, 80, 85, 90, 95, or 99 percent pure.

The invention will be further described in the following examples, which do not limit the scope of the invention described in the claims.

### EXAMPLES

### Example 1

### Anti-Aβ42 and Anti-Aβ40 Specific Monoclonal Antibodies Attenuate Amyloid Deposition in an Alzheimer's Disease Mouse Model

### Methods and Materials

Antibodies. The mAbs used for immunizations are shown in Table 1. The antibodies were generated as follows. Culture supernatants of hybridoma cells were screened for binding to Aβ immunogens by ELISA. Positive clones were then grown in suspension in DMEM medium, supplemented with 10% FCS Clone 1 and 1 mg/mL IL-6. Secreted antibodies were purified using Protein G columns and then used for all experiments. Mouse IgG was purchased from Equitech, Inc., Kerrville, Tex.

Mice. Tg2576 mice (B6/SJL, hAPP^{+/-}) were obtained from Charles River Laboratories (Wilmington, Mass.). To generate CRND8 mice, male CRND8 mice containing double mutation in human APP gene (KM670/671NL and V717F) (Chishti et al., J. Biol. Chem., 276:21562-21570 (2001)) were mated with female B6C3F1/Tac that were obtained from Taconic (Germantown, N.Y.). Genotyping of Tg2576 and CRND8 mice was performed by PCR as described previously (Hsiao et al., Science, 274:99-102 (1996) and Chishti et al., J. Biol. Chem., 276:21562-21570 (2001)). All animals were housed three to five to a cage and maintained on ad libitum food and water with a 12 hour light/dark cycle.

Capture ELISA for comparison of cross reactivity of end specific mAbs. Serial dilutions of Aβ40 and Aβ42 were used to determine the crossreactivity of Ab40.1 and Ab42.2. Ab9 was used as capture and Ab40.1-Horseraradish peroxidase (HRP) as a detection or Ab42.2 as a capture and Ab9-HRP as detection.

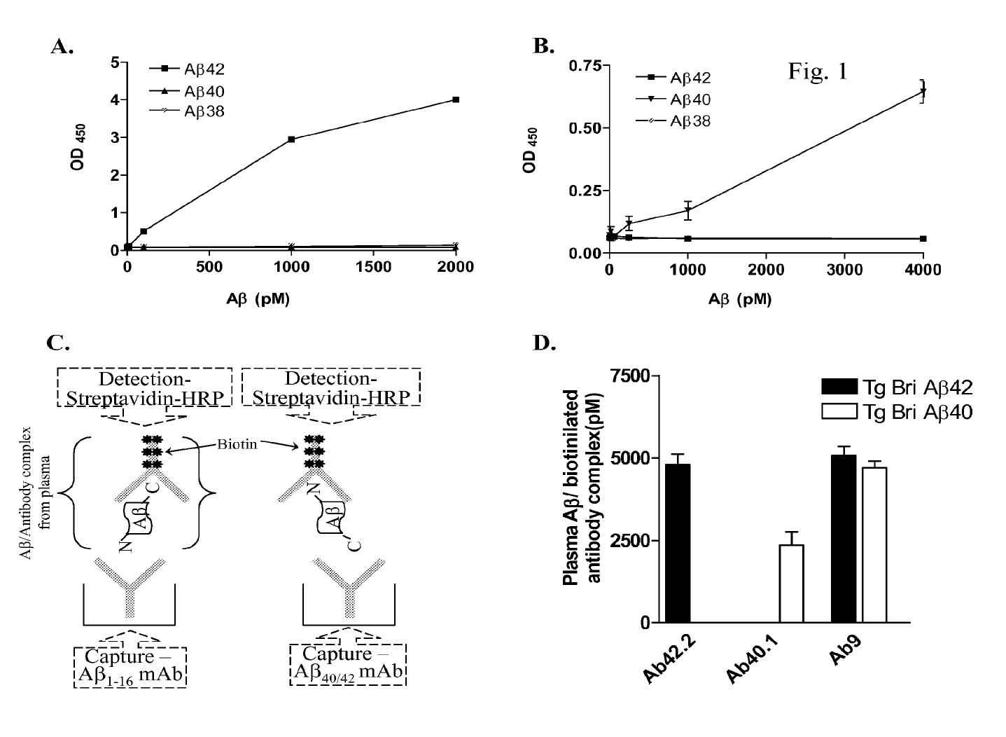

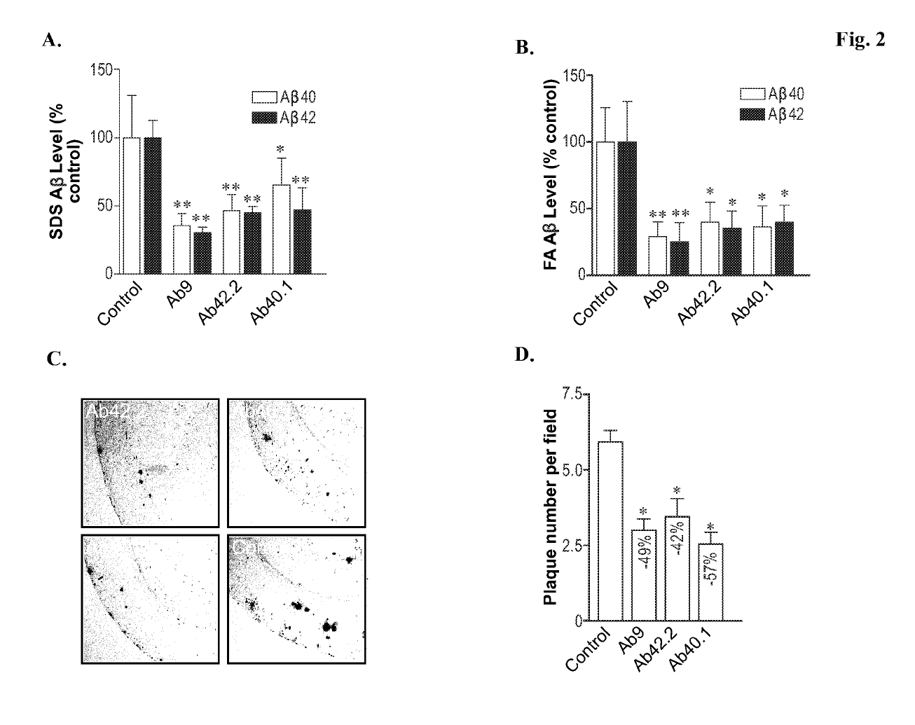

Measurement of Aβ-mAb complex in plasma. To measure the Aβ-biotinylated mAb complex in the plasma, TgBri-Aβ40 and TgBri-Aβ42 transgenic mice that express exclusively Aβ40 or Aβ42, respectively were immunized with 500 μg biotinylated mAb (i.p.) and plasma was collected 72 hours later. An mAb against the free end of Aβ peptide was used as capture and streptavidin-HRP as detection (FIG. 1c).

Staining of lightly fixed Aβ plaques. Cryostat sections (10 μm) from frozen unfixed human AD tissue (hippocampus) were lightly fixed in cold acetone for 2 minutes, blocked with 1% normal goat serum for 1 hour and then incubated with mAbs Ab9, Ab3, Ab2 or Ab5, each at 1 μg/mL, for 2 hours at room temperature. Slides were then washed in PBS, and incubated with goat-anti mouse conjugated to AlexaFluor-488 (1:1000, Molecular probes, Eugene, Oreg.) for 1 hour, washed, and mounted. For quantification of fluorescence, images of at least 3-5 randomly selected fields of plaques were obtained, and fluorescence intensity levels on individual plaques were measured using Analytical Imaging System (AIS, 4.0, Imaging Research, Ontario, Canada). The average fluorescent intensity level per plaque was determined by summing the fluorescent intensity of plaques divided by the total number of plaques analyzed (total of 10-15 plaques of equal size/group were used).

Passive immunizations. Groups of Tg2576 mice (females, 7, 10 or 11 month old, n=6/group) were immunized intraperitoneally (i.p.) with 500 μg of mAb once every 2 weeks for 4 months. CRND8 mice (females, 3 month old, n=7/group) were immunized intraperitoneally (i.p.) with 500 μg of mAb once every week for 8 weeks. Control mice received mouse IgG or PBS.

Cortical injections. For stereotaxic cortical injections, Tg2576 mice (females, 18 month old, n=3/group) mice were injected with 1 μg of the antibody in the frontal cortex of the right hemisphere whereas the left hemisphere was left untreated as a control. On the day of the surgery mice were anesthetized with isoflurane (5% initially and than 3% during the surgery) and placed in a stereotaxic apparatus. A midsagittal incision was made to expose the cranium and a hole was drilled to the following coordinates taken from bregma: A/P+1.1 mm, L-1.5 mm. A 26-gauge needle attached to a 10 μL, syringe was lowered 1.0 mm dorsoventral and a 2 μL, injection was made over a 10 minute period. The incision was closed with surgical staples and mice were then sacrificed 72 hours after the surgery.

ELISA analysis of extracted Aβ. At sacrifice the brains of mice were divided by midsaggital dissection, and one hemibrain used for biochemical analysis. Each hemibrain was sequentially extracted in a 2-step procedure as described elsewhere (Kawarabayashi et al., J. Neurosci., 21:372-381 (2001)). Briefly, each hemibrain (150 mg/mL wet weight) was sonicated in 2% SDS with protease inhibitors and centrifuged at 100,000×g for 1 hour at 4° C. Following centrifugation, the resultant supernatant was collected, representing the SDS-soluble fraction. The resultant pellet was then extracted in 70% formic acid (FA) and centrifuged, and the resultant supernatant collected (the FA fraction). The following antibodies against Aβ were used in the sandwich capture ELISA. For brain Aβ40, Ab9 was used as a capture antibody, and Ab40.1-HRP was used for detection. For brain Aβ42, Ab42.2 was uses as a capture antibody, and Ab9-HRP was used for detection.

Immunohistology. Hemibrains of mice were fixed in 4% paraformaldehyde in 0.1 M phosphate buffer (PBS, pH 7.6) and then stained for Aβ plaques as described elsewhere (Hardy and Selkoe, Science, 297:353-356 (2002) and Odaka et al., Neurodegenerative Diseases, 2:36-43 (2005)). Paraffin sections (5 μm) were pretreated with 80% formic acid for 5 minutes, washed and immersed in 0.3% of H₂O₂ for 30 minutes to block intrinsic peroxidase activity. They were then incubated with 2% normal goat serum in PBS for one hour, primary antibody (Monoclonal 33.1.1 (Aβ1-16 specific) at 1 μg/mL dilution overnight, and then with HRP-conjugated goat anti-mouse secondary antibody (1:500; Amersham Biosciences, Piscataway, N.J.) for one hour. Sections were washed in PBS, and immunoreactivity was visualized by DAB according to manufactures specifications (ABC system, Vector Labs, CA). Adjacent sections were stained with 4% thioflavine-S for 10 minutes. For cerebro-vascular amyloid detection, paraffin sections were stained with biotinylated Ab9 antibody (1:500) overnight at 4° C. and then immunoreactivity was visualized by DAB according to manufactures specifications (ABC system, Vector Labs, CA). Positively stained blood vessels in the neocortex were visually assessed and divided to a three groups relative to the severity of CAA. Vessels with more than 80% of the perimeter stained were given a highest score “+++”, partially stained vessels with 30-80% staining were given “++”, and only marginally stained vessels (less than 30% stained) were given “+”. Immunostained vessels were quantified in the neocortex of the same plane of section for each mouse (5-10 sections/mouse). Microhemorrage in the vessels was assessed by staining of ferric iron with Perls staining according to a standard protocol and by hematoxylin and eosin (H&E) stain (Racke et al., J. Neurosci., 25:629-636 (2005)).

Quantitation of amyloid plaque burden. Computer assisted quantification of Aβ plaques was performed using the MCID Elite software (Imaging Research, Inc, Ontario, Canada). Serial coronal sections stained as above were captured, and the threshold for plaque staining was determined and kept constant throughout the analysis. For analysis of plaque burdens in the passive immunization experiments, immunostained plaques were quantified (proportional area in old animals with vast deposition or plaque counts in young mice) in the neocortex of the same plane of section for each mouse (10-20 sections/mouse). In mice that were injected with mAb directly into the right hemisphere of the cortex, immunostained and Thio-S stained plaques were quantified as above specifically in the vicinity of the injection site (2 mm×2 mm area block). A total of 6-10 injection sites (2 mm×2 mm blocks) per treatment group were used for quantitation. An additional series of 30 sites (2 mm×2 mm blocks) from the left hemispheres of cortices of mice that were not injected were also quantified and used as control values for amyloid plaque burden. All the above analyses were performed in a blinded fashion.

Statistical analysis. One-way ANOVA analysis of variance followed by the Dunnett's Multiple Comparison Test was performed using the scientific statistic software GraphPad Prism version 3.

### Results

Selective in vivo binding by anti-A/β42 and anti-A/β40 mAbs. Multiple anti-Aβ mAbs (Table 1) were generated and characterized. Based on in vitro ELISA analysis of their binding properties, both the anti-Aβ42 antibody (Ab42.2) and the anti-Aβ40 antibody (Ab40.1) are highly selective for Aβx-42 and Aβx-40, respectively, whereas the antibodies that recognize the NH₂-terminal epitope of Aβ (Aβ1-16) bind both Aβ40 and Aβ42 as well as other Aβ peptides (e.g., Aβ37, 38, 39) (FIG. 1a,b). To determine if these antibodies maintain their selectivity for specific Aβ species in vivo, novel transgenic BRI-Aβ mice that selectively express either Aβ1-40 (TgBRI-Aβ40) or Aβ1-42 (TgBRI-Aβ42) were used. In these TgBRI-Aβ mice, Aβ can be detected both in the brain and plasma (McGowan et al., Neuron, 47:191-199 (2005)). To evaluate in vivo binding of these antibodies in TgBRI-Aβ mice, biotinylated Ab42.2, Ab40.1, or Ab9 (anti-Aβ1-16 mAb) were injected intraperitoneally (i.p.), and biotinylated mAb Aβ complexes detected using a modified sandwich ELISA protocol (FIG. 1c,d). Biotinylated Ab9-Aβ complexes were detected in the plasma of both TgBRI-Aβ40 and TgBRI-Aβ42. Biotinylated Ab42.2-Aβ complexes were detected only in plasma from TgBRI-Aβ42 mice and not in TgBRI-Aβ40 plasma, whereas biotinylated Ab40.1-Aβ complex were detected only in TgBRI-Aβ40 mice and not in TgBRI-Aβ42. No signal was detected in non-Tg mice injected with any of these biotinylated mAbs. These data demonstrate the in vivo specificity of Ab42.2 and Ab40.1 mAbs by demonstrating that they selectively bind their target Aβ species in vivo.

TABLE 1
Antibodies used for passive immunization

| | | | | Plaque | |
|---|---|---|---|---|---|
| Antibody | Immunogen | Isotype | Epitope^{a} | binding^{c} | Specificity^{d} |
| Ab42.2 | Aβ₃₅₋₄₂ | IgG1 | Aβx-42 | − | <0.1% |
| Ab40.1 | Aβ₃₅₋₄₀ | IgG1 | Aβx-40 | − | <0.1% |
| Ab2 | fAβ₁₋₄₂^{b} | IgG3 | Aβ1-16 | + | Pan Aβ |
| Ab3 | Aβ₁₋₁₆ | IgG1 | Aβ1-16 | +++ | Pan Aβ |
| Ab5 | fAβ₁₋₄₂^{b} | IgG2b | Aβ1-16 | ++ | Pan Aβ |
| Ab9 | fAβ₁₋₁₆ | IgG2a | Aβ1-16 | +++ | Pan Aβ |

^{a}Epitope mapping using various Aβ peptides was performed on each antibody.
^{b}Immunogen used was aggregated Aβ1-42.
^{c}The ability of the mAb to bind plaques was assessed by staining cryostat sections from unfixed frozen human AD hippocampus.
^{d}Binding to serial dilutions of Aβ40, Aβ42 and Aβ38 were used to determine the cross reactivity of mAbs Ab42.2 and Ab40.1 by capture ELISA.

Passive immunotherapy with an anti-Aβ42 and anti-A/β40 specific mAbs attenuates amyloid deposition in young Tg2576 mice. Having established the in vivo binding specificity of Ab42.2, Ab40.1, and Ab9, the effect of peripheral administration of these mAbs on Aβ deposition in Tg2576 mice was tested. Two studies were performed. A “prevention study” in which the anti-Aβ mAbs were administered to 7-month-old female Tg2576 mice which have minimal Aβ deposition, and a “therapeutic study” in which the mAbs were administered to 11-month-old Tg2576 that have moderate levels of pre-existing Aβ deposits (Kawarabayashi et al., J. Neurosci., 21:372-381 (2001)). Biochemical and immunohistochemical methods were used to analyze the effect of passive immunization on Aβ deposition (FIG. 2). After four months of passive immunization with Ab9, Ab42.2, and Ab40.1 initiated when the mice were 7-months-old, Aβ levels were significantly attenuated as assessed biochemically with Aβ ELISA following SDS extraction (>50% reduction in SDS Aβ, FIG. 2a) or formic acid extraction of the SDS-insoluble material (>50% reduction in FA Aβ, FIG. 2b). Representative immunostained sections are shown from immunized and control Tg2576 mice (FIG. 2c). Quantitative analysis of multiple immunostained sections also revealed a significant decrease in Aβ deposition. Both plaque numbers per field (FIG. 2d) and total immunoreactive plaque load were significantly reduced. The ratio between Aβ42 and Aβ40 was not significantly altered in either Ab42.2 or Ab40.1 treated mice. In contrast, four months of passive immunization with these same antibodies initiated when the Tg2576 mice were 11-months-old had no significant effect on biochemical (FIG. 2e and f) or immunohistochemical Aβ loads, although a slight but non-significant decrease in the SDS Aβ is seen in the Ab9 treated animals (35% reduction in SDS-Aβ, FIG. 2e).

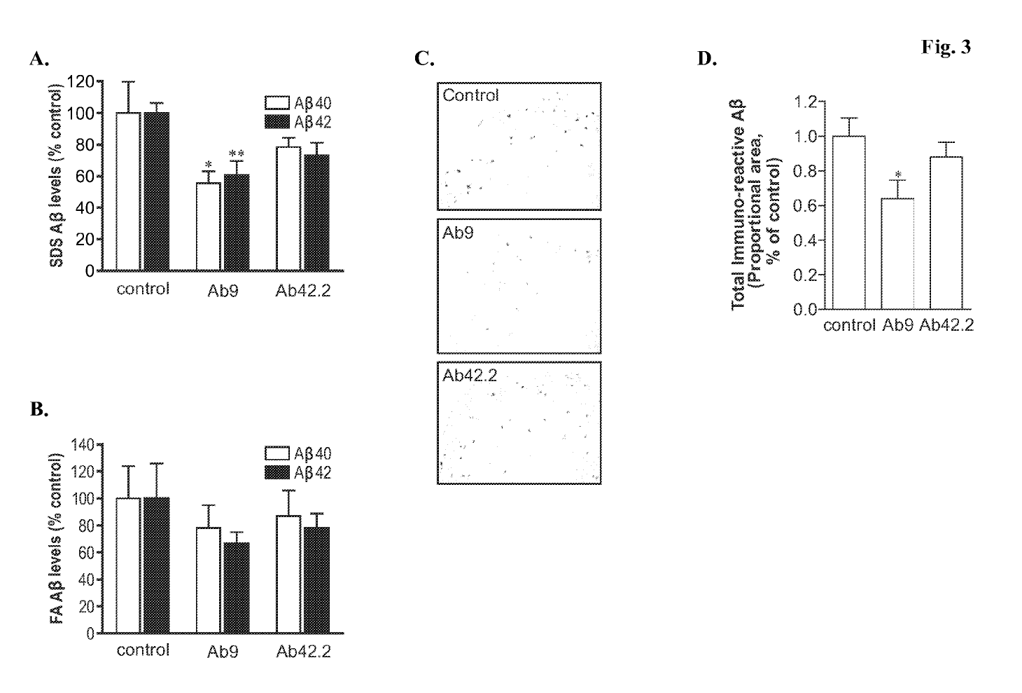

To further examine the relative efficacy of these anti-Aβ antibodies in altering Aβ accumulation, CRND8 mice were passively immunized. This transgenic model has a very early onset of Aβ deposition both as amyloid and in more diffuse plaques. Furthermore, compared to Tg2576 mice the relative level of Aβ42 is much higher then Aβ40 (Wang et al., Exp. Neurol., 158:328-337 (1999)). Thus, in CRND8 mice as in most cases of AD, the predominant species deposited is Aβ42. In contrast, Aβ40 is the predominant species deposited in Tg2576 mice. At 3 month of age, CRND8 mice have amyloid pathology that is roughly comparable to that of 10-month-old Tg2576 mice. Weekly injections of 3-month-old CRND8 mice with 500 μg of anti-Aβ Ab9 and Ab42.2 mAbs for 8 weeks resulted in significant reduction of SDS but not FA Aβ levels only in Ab9 treated mice (>40% reduction in SDS Aβ, FIG. 3a). Total Aβ42 levels (SDS+FA) were also significantly reduced by Ab9 treatment. Quantitative analysis of the immunostained sections also revealed a significant decrease in Aβ deposition in Ab9 treated mice (FIG. 3c and d). Immunization with Ab42.2 did not lead to a significant decrease in Aβ load, although there is a trend towards reduction in Aβ42 levels (p=0.13), suggesting that this antibody is less effective than Ab9 in clearing amyloid deposits in CRND8.

Effects on cerebral amyloid angiopathy (CAA) and CAA-related microhemorrhage. Passive immunization increases amounts of vascular amyloid staining in very old Tg2576 mice (Wilcock et al., J. Neuroinflammation, 1:24 (2004)). To examine the effect of passive immunization on CAA in the models described herein, brain sections were stained with biotinyated anti-Aβ mAb Ab9. Vessels with detectable CAA were divided into three groups relative to the extent of CAA within each vessel as visualized by immunostaining and the number of vessels with varying degrees of CAA counted in 5-10 sections per mouse. In Tg2576 mice, as well as in CRND8 mice, CAA was mostly associated with areas rich in amyloid plaques (Table 2), a result that is consistent with recent findings (Kumar-Singh et al., Am. J. Pathol., 167:527-543 (2005)). In 7-month-old Tg2576 mice immunized with anti-Aβ mAbs few blood vessels with trace amounts of Aβ amyloid staining were detected in control mice, but not in the immunized mice that have decreased levels of amyloid in the brain. Similarly, in the passively immunized CRND8 mice the number and the intensity of CAA-positive vessels were slightly but not significantly reduced (Table 2). The Tg2576 in the therapeutic study mice had extensive CAA in the neocortex. Following immunization, there was no appreciable difference in extent of CAA between control and treated mice. Passive immunization with mAbs directed against the NH2-terminus of Aβ has recently been reported to exacerbate CAA related microhemorrhage in PDAPP and APP23 transgenic mice (Racke et al., J. Neurosci., 25:629-636 (2005) and Pfeifer et al., Science, 298:1379 (2002)). Using both Perls stain and H&E to visualize microhemorrhages, no evidence for appreciable levels of microhemorrhage was found (less than one micohemorrhage event per brain section) in the control Tg2576 and CRND8 mice, nor was there a detectable increase in microhemorrhage following antibody administration.

TABLE 2
Effect of immunotherapy on number of CAA-positive blood vessels in the
neocortex of Tg2576 and CRND8 mice.

| | | +++^{a} | ++ | + |
|---|---|---|---|---|
| Tg2576 (preventative | control | 0^{b} | 0 | 2.4 ± 0.5 |
| study) | Ab9 | 0 | 0 | 0 |
| | Ab42.2 | 0 | 0 | 0 |
| CRND8 (therapeutic | control | 7 ± 0.8 | 4.4 ± 0.5 | 3.2 ± 0.9 |
| study) | Ab9 | 4.3 ± 0.5^{c} | 4 ± 0.9 | 2.7 ± 0.9 |
| | Ab42.2 | 6.3 ± 0.3 | 6 ± 0.9 | 4.3 ± 0.6 |
| Tg2576 (therapeutic | control | 3 ± 0.3 | 5.2 ± 1.6 | 6.3 ± 3.2 |
| study) | Ab9 | 2.6 ± 0.3 | 4.3 ± 1.9 | 7.2 ± 2.1 |
| | Ab42.2 | 2.8 ± 0.4 | 4.6 ± 1.3 | 7.8 ± 2.6 |

^{a}Positively stained for Aβ blood vessels were given a relative score: +++ full stain (>80% of vessel's perimeter stained), ++ partial stain (30-80%), + marginal stain (<30%).
^{b}Number of immunostained vessels in the neocortex (5-10 sections/mouse).
^{c}p < 0.05

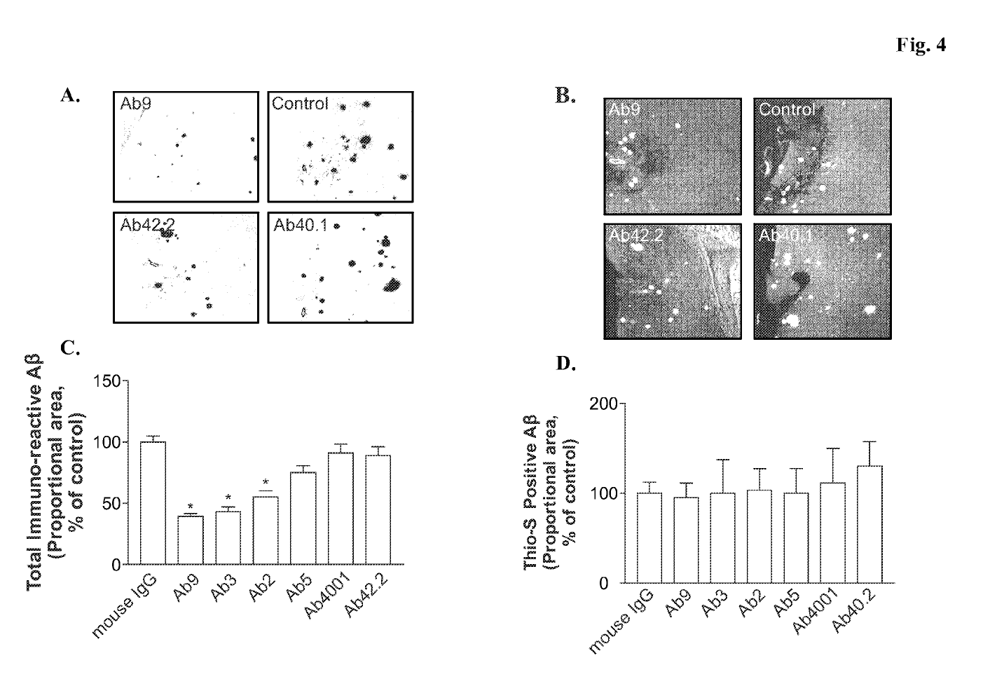

Direct cortical injections of anti-Aβ mAbs. To further explore the ability of the antibodies to alter plaque deposition, the effects of direct intracortical injections of the anti-Aβ40 and anti-Aβ42 mAbs and multiple anti-Aβ1-16 mAbs using 18-month-old Tg2576 mice were examined. In each case, 72 hours following cortical injection, the mice were killed, and the immunostained plaque load and Thioflavin S positive plaque load determined in the immediate vicinity of the injection site. Immunostained plaque load of Aβ was significantly decreased by three anti-Aβ1-16 mAbs (Ab9, Ab3 and Ab2), whereas the anti-Aβ1-16 mAb (Ab5) and both anti-Aβ40 and anti-Aβ42 mAbs had no measurable effect (FIG. 4a). In contrast, Thioflavin S staining of adjacent serial sections showed no effect on dense cored plaque loads with any mAb (FIG. 4b), suggesting that only diffuse Aβ deposits were selectively cleared by certain anti-Aβ1-16 antibodies. To confirm that control mouse IgG did not have any effect on plaque load, plaque load in control IgG treated sections was extensively compared with plaque load in the contralateral non-injected areas. There was no significant difference between them.

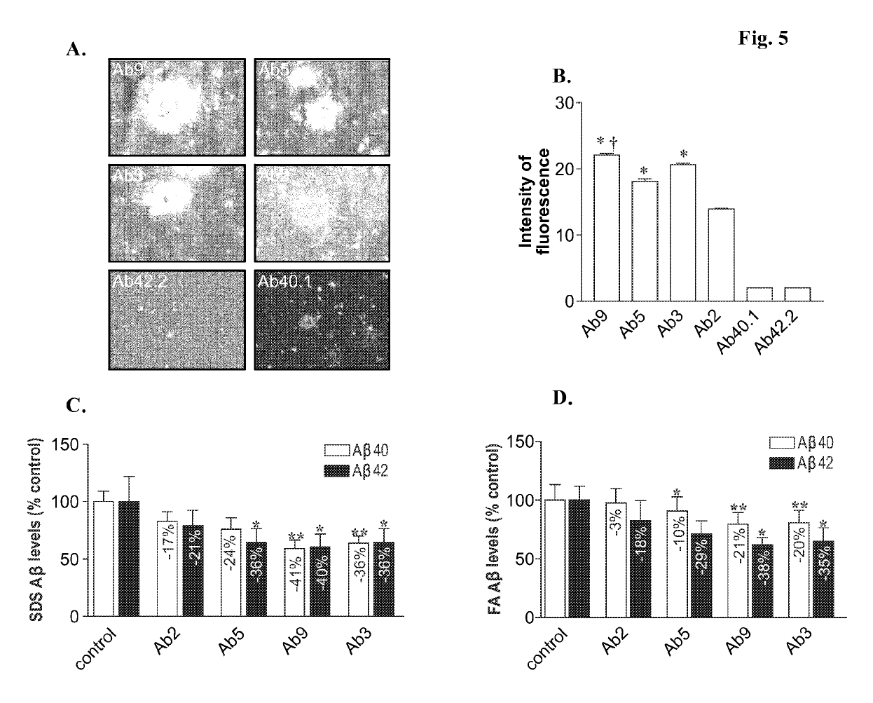

Binding of mAbs to plaques correlates well with their ability to alter Aβ deposition in mice with pre-existing Aβ deposits. In order to further characterize the properties of these mAbs associated with the ability to apparently clear preexisting diffuse Aβ deposits, two additional studies were performed. First, the relative affinity of these mAbs for binding to native unfixed plaques using frozen unfixed AD brain sections was compared (FIG. 5a). These data show that anti-Aβ40 and anti-Aβ42 antibodies did not bind native plaques, whereas all of the anti-Aβ1-16 antibodies show significant binding (FIG. 5a). Quantification of the fluorescent intensity per plaque did reveal that there were differences in the relative affinity for plaques based on this assay between the anti-Aβ1-16 antibodies (Ab9=Ab3>Ab5>Ab2) (FIG. 5b). Though neither anti-Aβ40 or anti-Aβ42 antibodies bind plaques in this assay, both antibodies do bind plaques following formic acid treatment of formalin fixed sections. Previous reports have implicated both native plaque binding and isotype as important determinants that correlated with efficacy of passive immunization with anti-Aβ mAbs (Bard et al., Proc. Natl. Acad. Sci. USA, 100:2023-2028 (2003)); therefore, the effect of each of the anti-Aβ1-16 mAbs on Aβ deposition in Tg2576 mice was compared. As noted above, these mAbs differ in their ability to recognize native amyloid plaques, but also encompass each of the four mouse IgG isotypes. In this study, immunization was initiated using 10-months-old Tg2576 mice and continued for four months. At sacrifice, biochemical Aβ loads were analyzed. Immunization with each mAb reduced SDS soluble Aβ levels (FIG. 5c). SDS-Aβ40 levels were significantly reduced by Ab9 and Ab3, and SDS-Aβ42 levels were significantly reduced by Ab9, Ab3, and Ab5. Similarly, FA Aβ was also reduced by each mAb (FIGS. 5d). Ab9 and Ab3 treatment resulted in significant reductions in both FA Aβ40 and FA Aβ42 levels, whereas only the reduction in FA Aβ40 was significantly reduced by Ab5. In this study, the rank order of efficacy of these four mAbs as passive immunogens correlated with their rank order in terms of plaque binding (Ab9=Ab3>Ab5>Ab2).

### Example 2

### Insights into the Mechanisms of Action of Anti-Aβ Antibodies in Alzheimer's Disease Mouse Models

### Methods

Antibodies. The anti-Aβ1-16 specific mAb9 (IgG2a) and mAb3 (IgG1) used for immunizations as well as anti-Aβ40 specific mAb40.1 (IgG1) and anti-Aβ42 specific mAb42.2 (IgG1) used for ELISAs were characterized herein. Biotinylation was performed according to the manufacturer. Briefly, 0.27 μmols of Sulfo-NHS-LC-Biotin (Pierce) were added to 2 mg mAb9 or mouse IgG and incubated for 2 hours at room temperature, followed by purification of labeled protein over desalting column. 4G8, human Aβ17-14 epitope was obtained from Signet (Dedham, Mass.). Mouse IgG was obtained from Equitech-Bio Inc.

Mice. Tg2576 mice and BRI-Aβ42B mice were generated and confirmed by genotyping. All animals were housed 3-5 to a cage and maintained on ad libitum food and water with a 12-hour light/dark cycle.

Binding kinetics. Affinity measurements were performed using a BIAcore X biosensor (BIAcore Inc., Piscataway, N.J.). A CM5 sensor chip (BIAcore) was activated as recommended by the manufacturer using an equimolar mix of NHS (N-hydroxysuccinimide) and EDC (N-ethyl-N′-(dimethylaminopropyl)carbodiimide), and immobilized with 50 μL of a capture antibody (BR100514, 100 μg/mL in 10 mM Na-acatate, pH 4.8), and then blocked with ethanolamine. 70 μL of the mAb (diluted in running buffer (HBS-EP) at 100 μg/mL) was injected onto the immobilized chip. The association and dissociation rate constants (kₐ and k_{d}) were determined using an Aβ concentration range with HBS-EP (0.01 M HEPES, 0.15 M NaCl, 3 mM EDTA, 0.005% (v/v) surfactant P20, pH 7) (BIAcore) as a running buffer at a flow rate of 10 μL/minute. The sensor surface was regenerated using 10 mM Glycine-HCl, pH 1.5. Kinetic parameters were evaluated using BIAevaluation 3.1 software (BIAcore).

Passive immunizations. Young Tg2576 mice or non-transgenic controls (3 month old, n=4 per group) were given a single i.p. dose of 500 μg (1600 pmol) biotinylated mAb9. Control mice received biotinylated mouse IgG or PBS.

Intracerebroventricular injections. For stereotaxic ICV injections, non-transgenic mice (females, 3 month old, n=2 per group) were injected with preformed complex of 50 μg (˜160 pmol) of biotinylated mAb9 and ˜320 pmoles of Aβ in the left cerebral ventricle. On the day of the surgery, mice were anesthetized with isoflurane (5% induction, 3% maintenance) and placed in a stereotaxic apparatus. A midsagittal incision was made to expose the cranium, and a hole was drilled to the following coordinates taken from bregma: A/P, −0.4 mm; L, −1.0 mm. A 26-gauge needle attached to a 10 μL, syringe was lowered 1.8 mm dorsoventral, and a 4 μL, injection was made over 10 minutes. The incision was closed with surgical staples, and the mice were sacrificed at various time points after the surgery.

Measurement of mAb9, Aβ or Aβ40: mAb9 complexes in plasma. Groups of female Tg2576 mice or their non-transgenic littermates were immunized with biotinylated mAb9, and plasma was collected at various time points. Control mice received biotinylated mouse IgG or PBS. To measure the Aβ40-biotinylated mAb9 complex in the plasma capture ELISA was used with an antibody against free end of Aβ40 peptide, mAb40.1 (2.5 μg/well), as capture and Neutravidin-HRP, 1:2000, as detection. For standards we saturated mAb40.1-coated plate with Aβ (5 μg/well), applied increasing amounts of biotinylated mAb9 and detected with Neutravidin-HRP. Control PBS injected plasma was spiked with 500 μg mAb9 to determine the basal levels of Aβ capable to bind mAb in the plasma. To determine the level of total Aβ40, mAb40 was used as a capture antibody, and 4G8, 1:2000, was used as a detection antibody. In non-Tg mice, levels of biotinylated mAb9 were determined by direct ELISA with Aβ40 (5 μg/well) as capture and Neutravidin-HRP as detection. Additionally, 1 mL plasma pooled from 3 mice 24 hours after the administration of biotinylated mAb9 or biotinylated mouse IgG was fractionated on a 1×30-cm Superose 6 PC 3.2/30 column (Amersham Biosciences). Superose columns were routinely pretreated with a bolus of BSA (50 mg) in running buffer to block nonspecific binding followed by a wash with at least 4 column volumes of running buffer. Aβ40 in each fraction was measured using capture ELISA as described above.

ELISA analysis of extracted Aβ from the brain. At sacrifice, the brains of mice were divided by midsagittal dissection, and both hemibrains were used for biochemical analysis. One hemibrain was homogenized in TBS with Complete™ protease inhibitors (150 mg/mL wet wt) while the other hemibrain was homogenized in RIPA (50 mM Tris-HCl pH 7.4, 150 mM NaCl, 1% Triton x-100, 1% Sodium deoxycholate, 0.1% SDS) with Complete™ protease inhibitor. Homogenates were than centrifuged at 20,000 g for 1 hour at 4° C., the resultant supernatant was collected, representing the TBS- or RIPA-soluble fraction, respectively. Additionally, a hemibrain was homogenized in Guanidinium Extraction Buffer (GuHCl, 5M Guanidine and 50 mM Tris-HCl) and incubated at room temperature for 4 hours, representing GuHCl fraction. The following mAbs against Aβ were used in the sandwich capture ELISA: for brain Aβ40, mAb40.1 capture and 4G8-HRP detection; for brain Aβ42, mAb42.2 capture and 4G8-HRP detection. To determine the amount of biotinylated mAb in the brain, direct ELISA with Aβ40 as capture and Neutravidin-HRP as detection was used.

Collection of cerebrospinal fluid. The procedure was performed according to that described elsewhere (Vogelweid et al., Laboratory Animal Science, 38, 91-92 (1988)). Briefly, mice were anesthetized with 2.5% Avertin IP. The animal's fur was clipped and placed in ventral recumbence over a gauze roll (attached to a 13×10×6 cm support) allowing the head to lie at a 45 degree angle. A small strip of transpore tape was used to hold the head in place. A midline incision starting at the base of the pinnae and continuing for approximately 1 cm caudal was made with a #10 blade. Iris scissors were used to separate the muscle layers of the “pocket” approximately 2 mm below the caudal edge of the occipital bone down to atlas. The underlying layers were bluntly separated with microdissecting forceps and retracted with bull clamps to visualize the dura mater, an opaque triangular-shaped membrane. If micro-hemorrhaging occurred during dissection, the window was blotted gently with an absorbent triangle to clear the area. An 18 gauge needle was guided to gently pierce the dura mater over the cisterna magna followed by immediate replacement with a pulled pipette (and aspirating bulb) to collect the CSF. The CSF was transferred to a gas tight screw cap vial and stored at −80° C.

Measurement of Aβ and A/β40-mAb complex in CSF. To measure the Aβ40-biotinylated mAb9 complex in the CSF capture ELISA was used with an antibody against free end of Aβ40 peptide, mAb40.1 as capture and Neutravidin-HRP as detection. To determine to level of total Aβ, mAb40.1 was used as capture and 4G8-HRP as detection.

Statistical analysis. One-way analysis of variance followed by the Dunnet's Multiple Comparison Test was performed using the GraphPad Prism version 4 software.

### Results

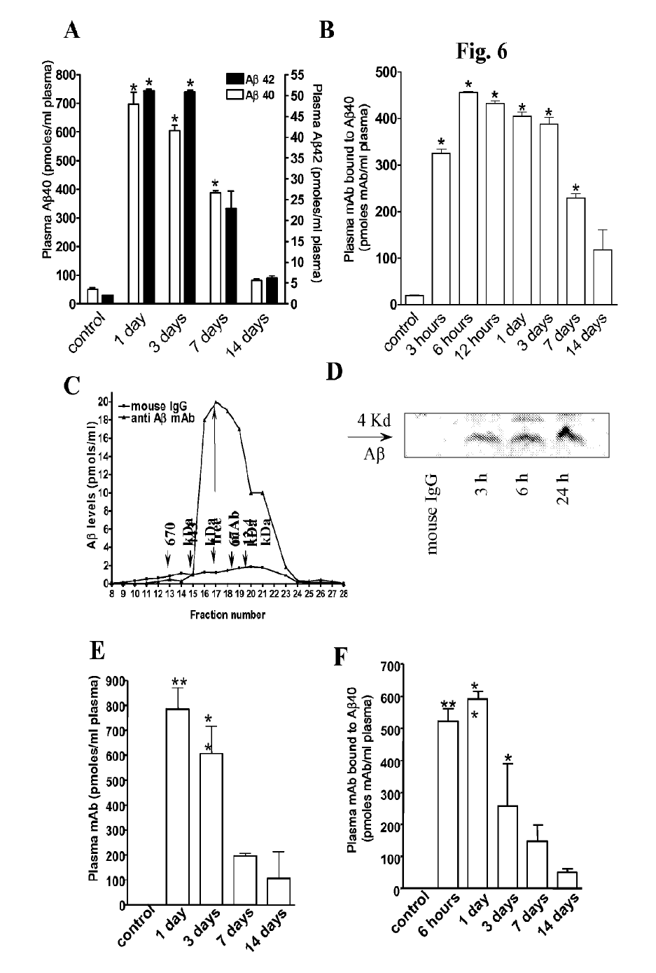

Peripheral administration of anti-Aβ/mAb creates a stable mAb:Aβ complex in the plasma. Aβ has a very short half-life in the plasma. When free Aβ is injected intravenously into the animal, it is cleared with a half-life of less than 10 minutes. Such data are consistent with findings that i.p. administration of a single 20 mg/kg of dose of a γ-secretase inhibitor to Tg2576 mice can reduce plasma Aβ by 80% within one hour and by greater then 98% within 5 hours, indicating that even endogenous plasma Aβ has a short half life. To study changes in Aβ levels induced by passive immunization with an anti-Aβ mAb as well as the in vivo binding properties and plasma half-life of the mAb itself, 500 μg (˜1600 pmoles) of biotinylated mAb9 was administered i.p. to 3 month old non-depositing female Tg2576 mice. Plasma Aβ levels were analyzed by capture ELISA over an extended time course. To insure that the biotinylated mAb9, which recognizes Aβ1-16, did not interfere with detection of Aβ by ELISA, Aβ was captured with end specific anti-Aβ mAbs and detected with HRP-conjugated 4G8, which recognizes a non-overlapping epitope on Aβ. In pilot studies with synthetic Aβ standards, mAb9 did not interfere with Aβ detection in end specific capture 4G8 detection ELISAs. Following biotinylated Ab9 administration, within 1 day after administration, Aβ40 in the plasma increased ˜15-fold, from ˜50 pmol/mL in untreated mice to almost 750 pmol/mL and Aβ42 levels increased ˜25-fold, from ˜2 pmol/mL in untreated mice to almost 55 pmol/mL, respectively. Plasma Aβ levels then slowly decreased over an extended period of time to near basal levels by 14 days (FIG. 6A). To examine the extent to which mAb binding of Aβ causes an increase in plasma Aβ, biotinylated mAb9:Aβ complexes were detected in plasma using a modified ELISA. The biotinylated mAb9:Aβ complex is captured with an Aβ40 specific mAb, and the complex detected with Neutravidin-HRP. The amount of the biotinylated mAb9:Aβ40 complex reached its highest value of ˜450 pmoles mAb9 bound to Aβ40 per mL of plasma after 6 hours (FIG. 6B). The complex appears to be quite stable with a half-life of about 7 days. Although the difference in standardization methods between ELISA measurements of plasma Aβ and plasma biotinylated mAb9:Aβ complexes introduce some uncertainty with respect to the levels of “total” plasma Aβ relative to the level of biotinylated mAb9:Aβ, a comparison of the peak levels of total Aβ and mAb:Aβ complex would suggest that the majority of Aβ is bound to the mAb. Consistent with these data, preclearing the plasma with protein A/G removes over 90% of the ELISA signal. Size exclusion column chromatography of mouse plasma collected 1 day post mAb9 injection shows that most of the plasma Aβ that accumulates following mAb9 treatment is present in a high molecular weight fraction with a peak levels in a fraction that corresponds to the peak fraction in which unbound mAb9 elutes (FIG. 6C). In the plasma from the mice injected with biotinylated mouse IgG, the levels in most fractions are much lower, and Aβ appears to be broadly distributed presumably because, as previously reported, it is bound to numerous serum proteins. Finally, when plasma from biotinylated mAb9 injected mice is precipitated with Streptavidin beads and subjected to Western blot analysis, an increase in a 4 kDa Aβ species is observed (FIG. 6D).

The half-life of an IgG2a antibody in mouse plasma has been reported to be ˜1 week (Vieira et al., Eur. J. Immunol., 16, 871-874 (1986)). When 500 μg (˜1600 pmoles) of biotinylated mAb9 are administered to 3 month old female non-transgenic littermates of the Tg2576 mice, ˜800 pmol mAb9/mL plasma can be detected in the plasma 1 day later. The biotinylated mAb9 is quite stable and appears to have a half-life of 5-7 days (FIG. 6E). Collectively, such data suggest that the increase in Aβ levels is attributable to binding and stabilization of Aβ by the anti-Aβ mAb. To directly determine if binding of the mAb9 to Aβ prolongs the half-life of Aβ, a preformed complex of biotinylated mAb9 (500 μg, ˜1600 pmoles) and human Aβ40 (˜3200 pmoles) was administered via i.p. injection into young non-transgenic mice. The mAb9:Aβ40 complex was detected as described previously. As mAb9 does not recognize mouse Aβ; these studies are not confounded by mAb interaction with endogenous mouse Aβ. Within 6 hours, about 500 pmoles/mL of the complex are detected and the complex, like the unbound antibody, is cleared slowly with a half-life of ˜5-7 days (FIG. 6F). Thus, in contrast to endogenous Aβ, the mAb9:Aβ40 complex has a prolonged half-life. In addition, these studies would suggest that the binding of the mAb to Aβ does not result in the formation of a classic immune complex that would be rapidly cleared. Finally, such data suggest that in plasma the tight binding of mAb9 to Aβ (Kd is estimated by surface plasmon resonance to be ˜3.5e-9 M) prevents the bound Aβ from being rapidly turned over.

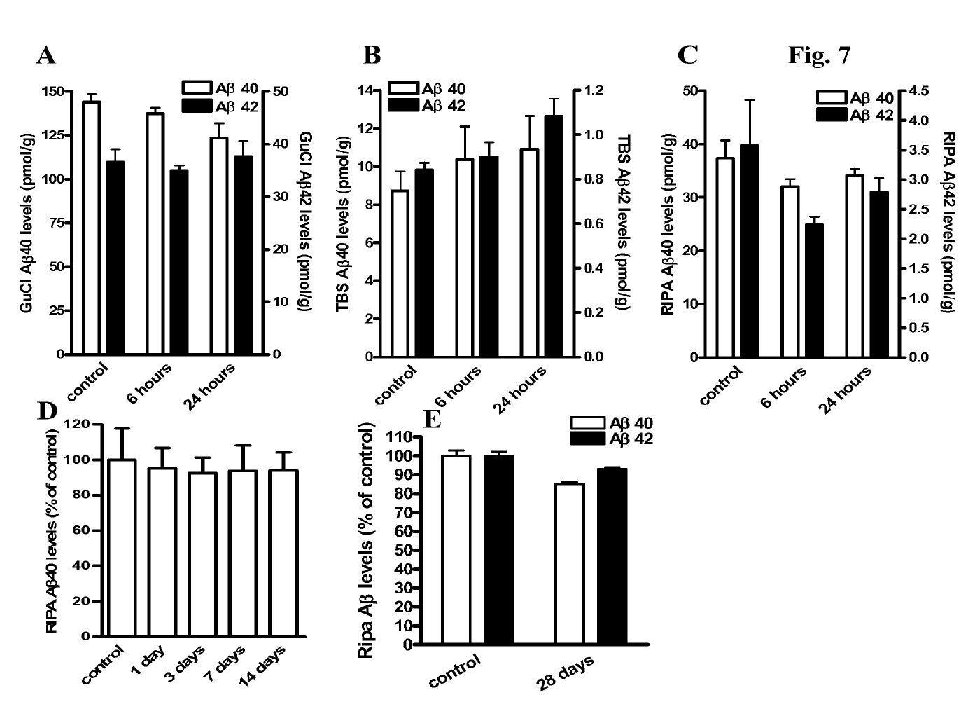

Effects of acute immunization with anti-A/β mAb on Aβ levels in the brains of Tg2576 and BRI-042B mice. To determine if alterations in brain Aβ occur following peripheral immunization, the effects on brain Aβ in young female Tg2576 mice were examined for up to two weeks following i.p. administration of 500 μg of biotinylated mAb9. To reduce interference from vascular Aβ and mAb9, the mice were extensively perfused with PBS prior to brain harvest. Aβ40 and Aβ42 levels were measured by ELISA in separate TBS, RIPA, and 5M Guanadinium hydrochloride (GuHCl) fractions. In these studies and as previously reported, GuHCL extracts the highest levels of Aβ from the brain, and despite the marked accumulation of plasma Aβ at the 6 and 24 hour time points, there is no appreciable change in the levels of GuHCl-extractable brain Aβ40 or Aβ42 (FIG. 7A). TBS extracts presumably reflect levels of soluble Aβ, and contain much lower amounts of Aβ than are present in the GuHCl extract (Aβ40˜6-7% and Aβ42˜2-3% of GuHCL extract Aβ levels). TBS-extractable Aβ40 and Aβ42 increase slightly following peripheral administration, though the absolute level of increase is small, ˜5-10% of control values, and does not reach statistical significance by ANOVA (FIG. 7B). RIPA, a moderately denaturing detergent mix, extracts a higher level of Aβ40 and Aβ42 than TBS but lower levels than GuHCl (Aβ40˜25-30% and Aβ42˜8-10% of the GuHCL extract Aβ levels). RIPA-extractable Aβ decreases slightly following immunization by 20% of control or ˜10 pmol/g (FIG. 7C). No statistically significant decrease in RIPA-soluble Aβ40 levels is detected up to 14 days after the single mAb administration (FIG. 7D). Moreover, the slight decrease observed 24 hours after the single mAb administration is not additive, since continuous weekly administration of 500 mg mAb for 4 weeks results in similar slight but not significant decrease in RIPA-soluble Ab levels (FIG. 7E).

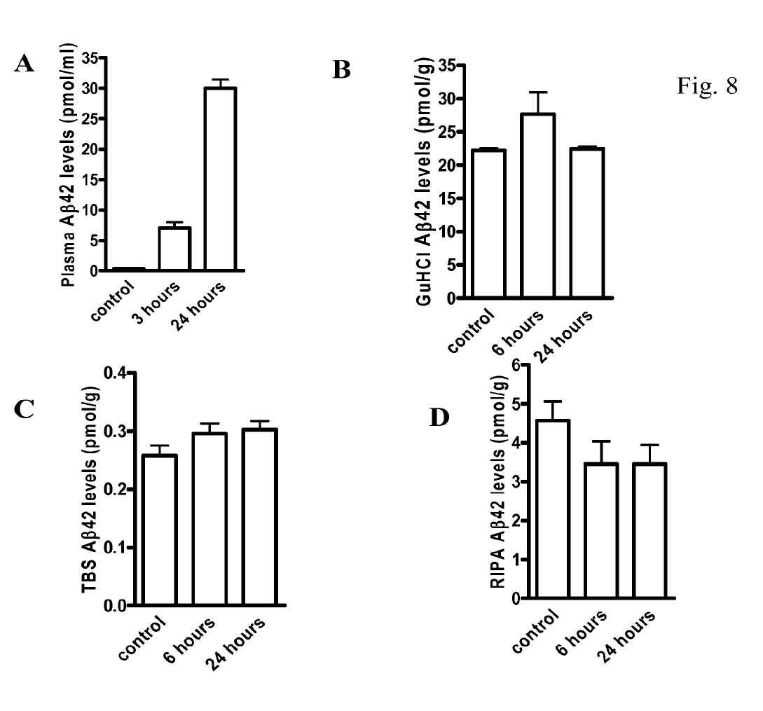

Tg2576 mice make large amounts of Aβ both peripherally and in the brain. In non-depositing Tg2576 mice, this Aβ is rapidly turned over. The half-life of Aβ in brain is estimated to be 1-2 hours. Indeed, studies on mAb9 binding to plasma Aβ in Tg2576 mice suggest that following peripheral immunization mAb9 is saturated with Aβ within 6-12 hours of administration. Thus, the small changes in brain Aβ observed in Tg2576 mice, immediately following mAb9 administration might be amplified if more mAb were administered or if the same amount of mAb was administered to a transgenic mouse which produces much lower levels of Aβ. Because an amount of mAb that was near the maximal tolerated dose was already being delivered, the same amount of biotinylated mAb9 was administered to a low expressing BRI-Aβ42B line. This line of BRI-Aβ42B mice only expresses Aβ42, and has ˜5-fold lower levels of total brain Aβ and ˜100 fold lower plasma levels relative to Tg2576 mice. At three months of age, these mice do not have detectable Aβ deposits. Following mAb9 administration, a rapid increase in Aβ levels was observed in the plasma from ˜0.5 pmol/mL in untreated mice to ˜7 pmol/mL at 3 hours and ˜30 pmols/mL 1 day after immunization (FIG. 8A). The amount of the biotinylated mAb9:Aβ42 complex increases in parallel. There was no significant change in total brain Aβ42 levels extracted by GuHCl (FIG. 8B), a slight non-significant increase in TBS-extractable brain Aβ42 levels (total increase ˜15%) (FIG. 8C) and a slight non-statistically significant decrease in RIPA-extractable Aβ42 (−25%) (FIG. 8D). The magnitude of these changes are similar to those seen in Tg2576 mice, indicating that the small effects induced by mAB9 administration are not influenced to any great extent by the relative amount of plasma or brain Aβ in the different transgenic lines.

Brain levels of mAb9 following acute peripheral administration of anti-Aβ mAb. In previous studies, we have failed to detect anti-Aβ mAb binding to plaques following peripheral anti-Aβ mAb administration using immunohistochemical techniques. Others, however, have reported that consistent with previous reports of blood brain barrier (BBB) penetrance of Abs that a small fraction of anti-Aβ mAbs can penetrate the BBB (if quantified levels are less than 0.1% of total dose; Bard et al., Nat. Med., 6:916-919 (2000), DeMattos et al., Science, 295:2264-2267 (2002), and Banks et al., Peptides, 26:287-294 (2005)). Following administration via i.p. injection of 500 μg (1600 pmoles) biotinylated mAb9 to non-transgenic mice, 1.0˜0.08 fmol/mg of biotinylated mAb9 was detected 6 hours post-injection, which is approximately ˜300 fmoles per brain or ˜0.02% of the total amount of the antibody administered. The levels of antibody fall by 24 hours to 0.53˜0.06 fmol/mg and by 2 weeks the levels are 0.06˜0.01 fmol/mg. Even lower levels of mAb9 were detected in the Tg2576 brain. Despite extensive perfusion, it is impossible to determine whether these trace amounts of mAb9 are truly in the brain or simply stuck to the cerebral vessels; multiple attempts to detect the mAb in situ in the brain sections using immunohistochemical techniques gave negative results. In any case, such data place an upper limit on the amount of mAb9 present in the brain at the time the plasma mAb levels are near maximal.

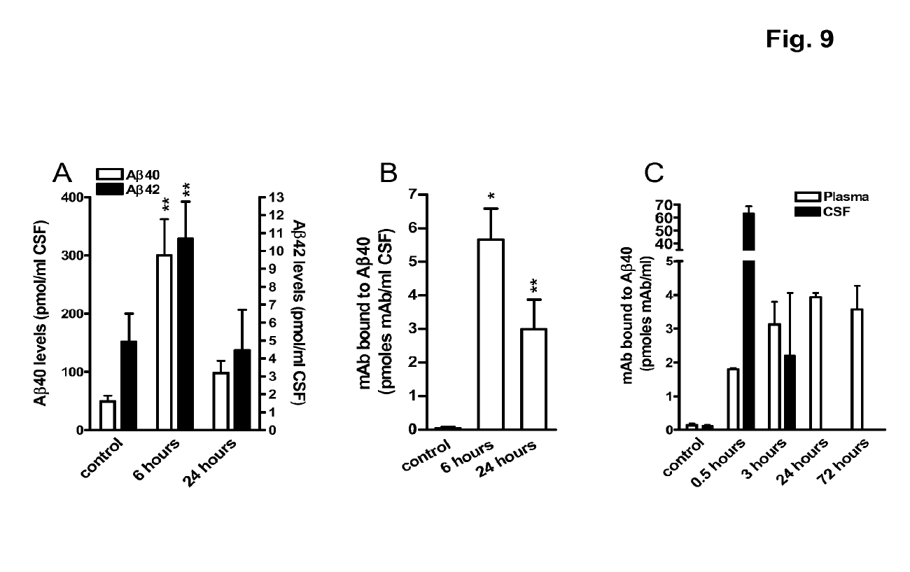

Effects of anti-Aβ mAb on CSF Aβ and clearance of mAb9:Aβ complexes from the brain. The levels of Aβ and biotinylated mAb9:Aβ complexes in the CSF following i.p. administration of mAb9 to Tg2576 mice were examined. Six hours post mAb injection, a 6-fold increase in Aβ40 and 2-fold increase in Aβ42 levels was observed in CSF collected from the cisterna magna. This result contrasts with plasma Aβ levels which peak at 6 hours post mAb injection and remain at a relatively stable baseline over 24-72 hours (FIG. 6A). CSF Aβ levels decrease rapidly towards control levels by 24 hours (FIG. 9A). Low levels of biotinylated mAb9:Aβ complexes are also detected in the CSF and change in parallel with Aβ levels (FIG. 9B). Unlike in plasma, where Aβ levels are roughly comparable to the levels of mAb9 bound to Aβ, in CSF there is ˜50 fold more Aβ than mAb bound to it. One possible explanation for this high ratio of Aβ to mAb, would be that mAb is bound to an Aβ aggregate in CSF. The concentration of the mAb9:Aβ complex in the plasma remain unchanged during this time period, suggesting that there may be rapid export of the mAβ9:Aβ complex from the CSF. To explore this possibility, a preformed complex of 5 μg (˜160 pmol) of biotinylated mAb9 and ˜320 pmoles of Aβ was injected ICV. Following injection into the ventricles, the biotinylated mAb9:Aβ complex is detected in CSF collected from the cisterna magna within 30 minutes. By 3 hours, the levels dramatically decreased, and at 24 hours no complex was detectable (FIG. 9C). In contrast, the low levels of complex appeared in plasma by 30 minutes and appeared relatively stable up to 72 hours post injection. Such data suggest that even though the anti-Aβ mAb:Aβ complex has a long-half life in the plasma, the complex is rapidly cleared from the CSF, and at least some of this clearance is via export into the vasculature.

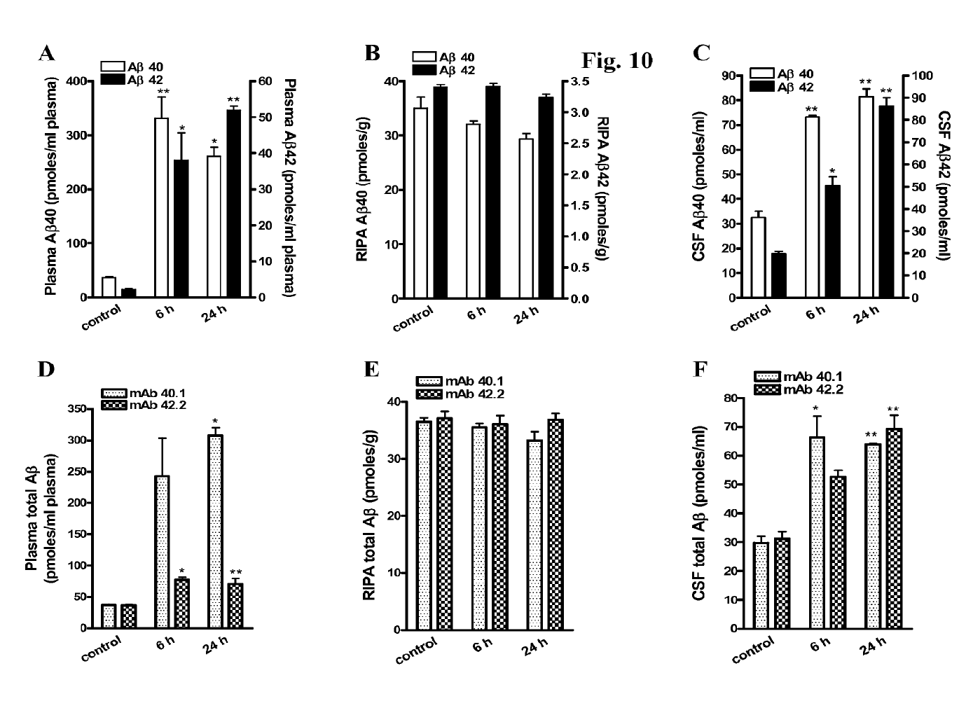

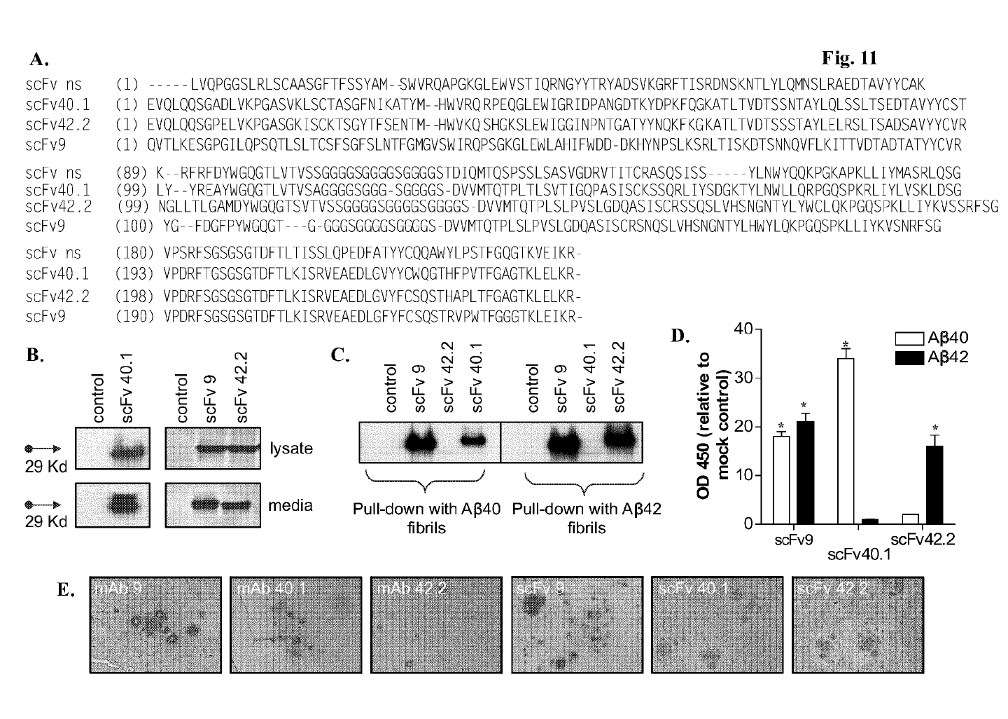

Additional anti-Aβ mAbs have similar effects on Aβ levels in plasma, brain and CSF of Tg2576 mice. To determine if the observed dynamics in plasma, CSF and brain following an acute dose of mAb in TG2576 mice are common to the other anti-Aβ mAb characterized in previous studies and shown to reduce Aβ deposition following peripheral administration, 500 μg biotinylated anti-Aβ1-16 mAb3, anti-Aβ42 mAb 42.2, and anti-Aβ40 mAb40.1 were injected to 3-month old Tg2576 mice. Like mAb9, mAb3 administration resulted in ˜7 fold increase in Aβ40 and ˜20 fold increase in Aβ42 levels in plasma (FIG. 10A), but only a slight non-significant decrease in Aβ40 levels in RIPA-soluble brain extracts and no effect on RIPA-soluble Aβ42 levels (FIG. 10B). mAb40.1 and mAb42.2 are end-specific antibodies that have been shown to selectively bind Aβ40 and Aβ42, respectively, in vivo. To avoid interference by the end-specific mAbs present in the plasma, in the ELISAs, total Aβ levels were only measured using mAb9 as capture and mAb 4G8-HRP as detection. Both end-specifc mAbs caused an increase in total Aβ levels in plasma 6 and 24 hours after the injection. Higher levels of plasma Aβ accumulated following administration of mbA0.1 then mAb42.2, presumably because the mAbs are end-specific and thus the “total” Aβ level reflects the relative abundance of these species in the plasma. No effect was observed on the brain RIPA-soluble Aβ (FIGS. 10D and E). Aβ levels in CSF were also increased upon administration of all three mAbs, although the dynamics of this increase vary between the antibodies (FIGS. 10C and F).

### Example 3

### Intracranial AAV Mediated Delivery of Anti-Pan Aβ, Aβ40 and Aβ42 scFvs Attenuates Plaque Pathology in APP Mice

### Methods

AAV construction and preparation. AAV was prepared by standard methods. Briefly, AAV vectors expressing the scFv under the control of the cytomegalovirus enhancer/chicken beta actin (CBA) promoter, a WPRE, and the bovine growth hormone polyA were generated by plasmid transfection with helper plasmids in HEK293T cells. 48 hours after transfection, cells were harvested and lysed in the presence of 0.5% Sodium Deoxycholate and 50 U/ml Benzonase (Sigma) by freeze thawing, and the virus isolated using a discontinuous Iodixanol gradient, and affinity purified on a HiTrap HQ column (Amersham). The genomic titer of each virus was determined by quantitative PCR.

Mice. To generate CRND8 mice, male CRND8 mice containing double mutation in human APP gene (KM670/671NL and V717F) (Chishti et al., J. Biol. Chem., 276:21562-21570 (2001)) were mated with female B6C3F1/Tac that were obtained from Taconic (Germantown, N.Y.). Genotyping of Tg2576 and CRND8 mice was performed by PCR as described herein. All animals were housed three to five to a cage and maintained on ad libitum food and water with a 12 hour light/dark cycle.

mRNA isolation, cDNA synthesis, amplification of cDNAs encoding V_{H} and V_{I}, regions, and construction of scFvs. mRNA was isolated from hybridomas cell lines using a mRNA isolation kit (Qiagen). cDNA was synthesized using MMLV Reverse Transcriptase (Promega) and random hexamers. The cDNA was than polyG-tailed with Terminal Transferase (NE BioLabs). cDNAs encoding the variable heavy (V_{H}) and variable light (V_{L}) chains were amplified using anchor PCR with a forward poly-C anchor primer and a reverse primer specific for constant region sequence of IgG2a (for pan Ab) and IgG1 for Ab40.1 and Ab42.2, as described elsewhere (Gilliland et al., Tissue Antigens, 47:1-20 (1996)). PCR products were than sequenced using the same primers, and the consensus V_{H} and V_{L} were determined. cDNAs encoding scFvs of three anti-Aβ antibodies were constructed by ligating the V_{H} and V_{L} cDNAs in V_{H}-linker-V_{L} orientation separated by Gly₄Ser₃ linker. Non-specific scFv (scFv ns) was randomly obtained from a phage library (Medical Research Council, Cambridge, England) and showed no affinity to Aβ.

Fibrillar Aβ pulldown assays. One mL of conditioned media from 293T HEK cells transiently transfected with pSecTag palsmids encoding the anti-Aβ scFv was incubated with 10 μg of fibrillar Aβ40 or Aβ42 (fAβ) at 4° C. for 1 hour. The fibrils were then spun down and resuspended in SDS-PAGE loading buffer. The presence of scFv was determined by western blot with rabbit anti-His (Bethyl). To determine the Aβ40 binding properties of scFv secreted into the media, capture ELISA was used with Aβ40 peptide as capture and anti-myc-HRP, 1:2000, as detection.

Neonatal injections. The procedure was adapted from that described elsewhere (Passini and Wolfe, J. Virol., 75:12382-12392 (2001)). Briefly, P0 pups were cryoanesthetized on ice for 5 minutes. 2 μL of AAV-scFv were injected ICV into the both hemispheres using a 10 mL Hamilton syringe with a 30 g needle. The pups were then placed on a heating pad with their original nesting material for 3-5 minutes and returned to their mother for further recovery.

Analysis of Aβ in the brain. The following antibodies against Aβ were used in the sandwich capture ELISA: For brain Aβ40-Ab9 capture and Ab40.1-HRP detection. For Brain Aβ42-Ab42.2 capture and Ab9-HRP detection. Biochemical Aβ analysis and immunohistochemical analyses were performed as described herein.

Measurement of Aβ-scFv complex in plasma. To measure the Aβ-scFv complex in the plasma of CRND8 mice 3 months following neonatal ICV injection of AAV-scFv, ELISA was performed with a mAb against the free end of Aβ peptide as capture (for scFv9-mAb40.1, for scFv40.1 and scFv42.2-mAb9) and anti-myc-HRP as detection.

Statistical analysis. One-way ANOVA analysis of variance followed by the Dunnett's Multiple Comparison tests were performed using the scientific statistic software GraphPad Prism version 4.

### Results

Construction and characterization of the scFvs. scFvs were cloned from hybridomas expressing an anti-Aβ1-16 mAb9 (IgG2ak), anti-Aβ40 specific mAb40.1 (IgG1k), and anti-Aβ42 specific mAb42.2 (IgG1k). The parent antibodies exhibited high specificity for Aβ, recognize amyloid plaques, and effectively attenuate amyloid deposition when administered to young Tg2576 mice. The amino acid sequences of scFv9, scFv40.1, scFv42.2 (derived from the anti-Aβ1-16 mAb9, the Aβx-40 specific mAb40.1, and the anti-Aβx-42 specific mAb42.2) are shown in the FIG. 11A along with a non-Aβ binding scFv (scFv ns) used as a control.

Prior to testing the effects of the scFv in vivo, the anti-Aβ scFvs expressed from 293T cells were characterized. Anti-AP scFvs were detected both in the 1% Triton cell lysate and in the conditioned media following transient transfection (FIG. 11B). The ˜28 kDa band detected on SDS-PAGE gel with an anti-His antibody represents monomeric scFvs secreted from the cells. ScFvs were also visualized in the cell by immunocytochemistry with an anti-6×His antibody. To show that the scFv bind Aβ, a fibrillar Aβ (fAβ) pulldown assay was used. Following fAβ42 pulldown, a ˜28 kDa band was detected from the conditioned media of cells transfected with scFv9 and scFv42.2, but not scFv40.1; whereas following fAβ40 pulldown a 28 kDa band was detected from the conditioned media of cells transfected with scFv9 and scFv40.1, but not scFv42.2 (FIG. 11C). In addition, when conditioned media was loaded on an Aβ40 coated ELISA plate and the bound scFv detected with HRP-conjugated anti-myc antibody, the media from scFv9 and scFv40.1 transfected cells exhibited a significant signal. When the same media was administered to an ELISA plate coated with Aβ42, a significant signal was only seen from scFv9 and scFv42.2 secreting cells (FIG. 11D), confirming the pulldown data. These scFvs were also able to detect amyloid plaques on paraffin sections from brains of old Tg2576 mice (FIG. 11E). Collectively, these data demonstrate that the three anti-AP scFvs maintain the binding properties of the parent mAbs.

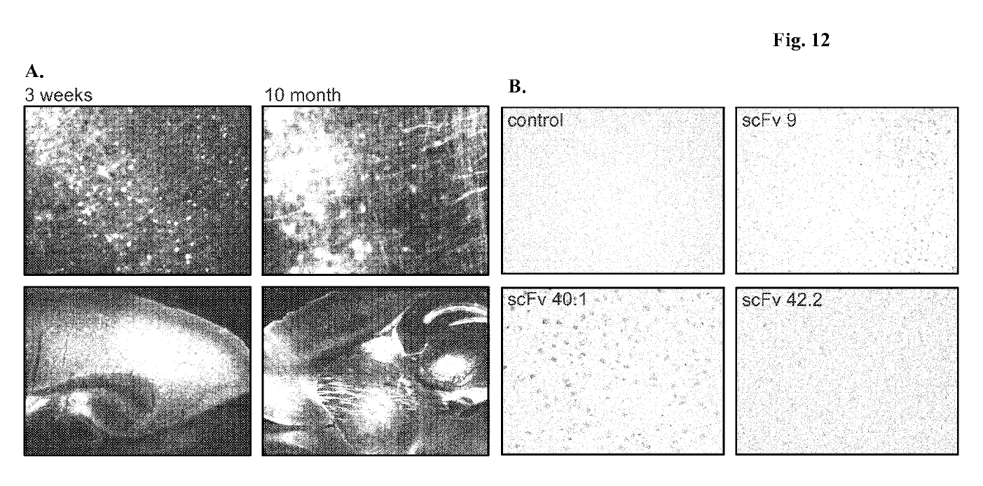

Intracranial expression of GFP and anti-Aβ scFv using AAV1 transduction of the neonatal brain. Injection of AAV serotype 1 (AAV1) into the cerebral ventricles of newborn mouse pups has been reported to result in widespread neuronal transduction and life-long expression of the packaged gene (Passini et al., J. Virol., 77:7034-7040 (2003)). AAV1 encoding hGFP (2×10¹⁰ genome particles/ventricle) was bilaterally injected into the cerebral lateral ventricles of P0 Swiss-Webster mice. GFP expression was detected by green fluorescence at three weeks as well as 10 months post injection (FIG. 12A). The most striking expression was seen in the neuronal cell layers of hippocampal CA1 to CA3 region, choroid plexus, and ependymal cells lining the ventricle. hGFP positive signals were also detected in periventricular areas and frontal cortex. Injection of 10-fold higher titers of AAV1-hGFP (total 4×10¹¹ genome particles) resulted in localized green fluorescence in choroid plexus and single layer of cells around the ventricle. In pups injected at P1 or P2, the transduction of AAV1 as visualized by hGFP expression was dramatically reduced with expression localized to the periventricular region. GFP expression was more readily detected in the neuronal cell bodies three weeks post injection, but redistributed into the neuronal processes by 10-months of age. No toxic side effects or post-operational mortality were observed in CRND8 mice injected with AAV1-hGFP at any stage of the experiment.

After confirming the ability of AAV1 to mediate widespread delivery of a transgene to P0 mouse pups, newborn P0 CRND8 mice as well as non-transgenic littermates were injected with AAV1 vectors encoding the various anti-Aβ scFvs (2×10¹⁰ genome particles/ventricle). Three weeks after the injection, scFv expression was detected by immunohistochemistry with anti-His antibody throughout the brain (FIG. 12B). The distribution of each anti-Aβ scFv was similar to each other and to hGFP, demonstrating that widespread delivery of the transgene was achieved using AAV1 vectors. Cell body staining was noted in spite of scFv being a secreted protein, as well as a general increase in the background, possibly attributable to the presence of scFv in processes or to secreted scFv.

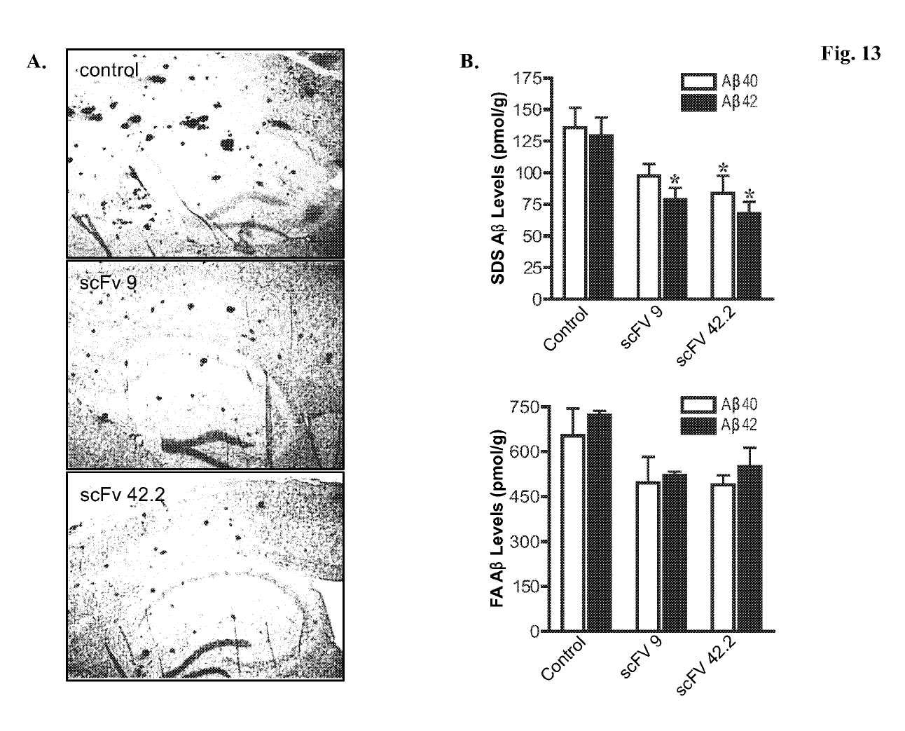

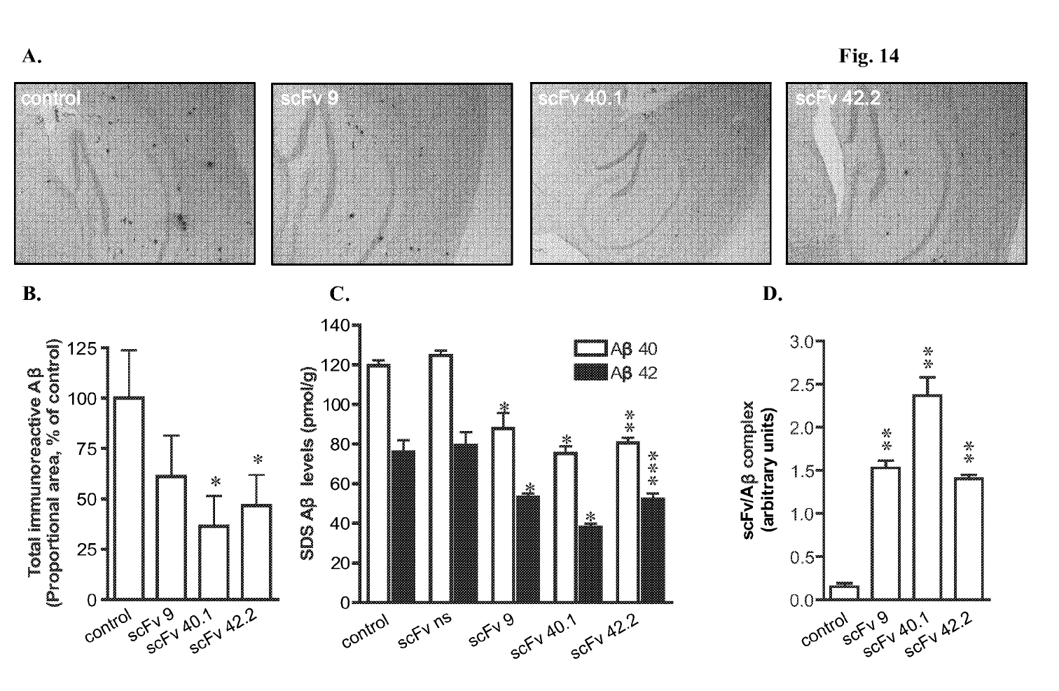

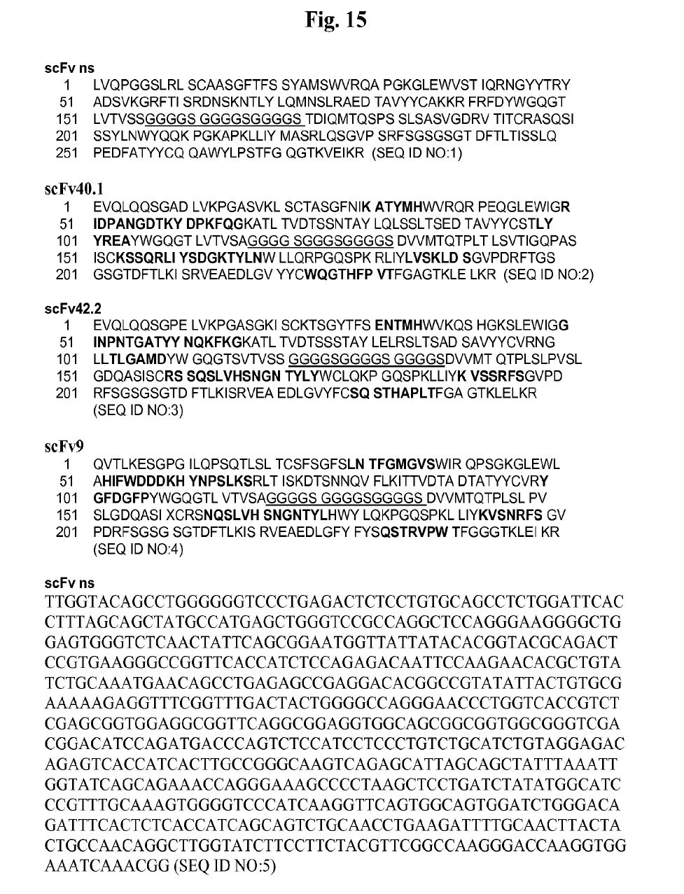

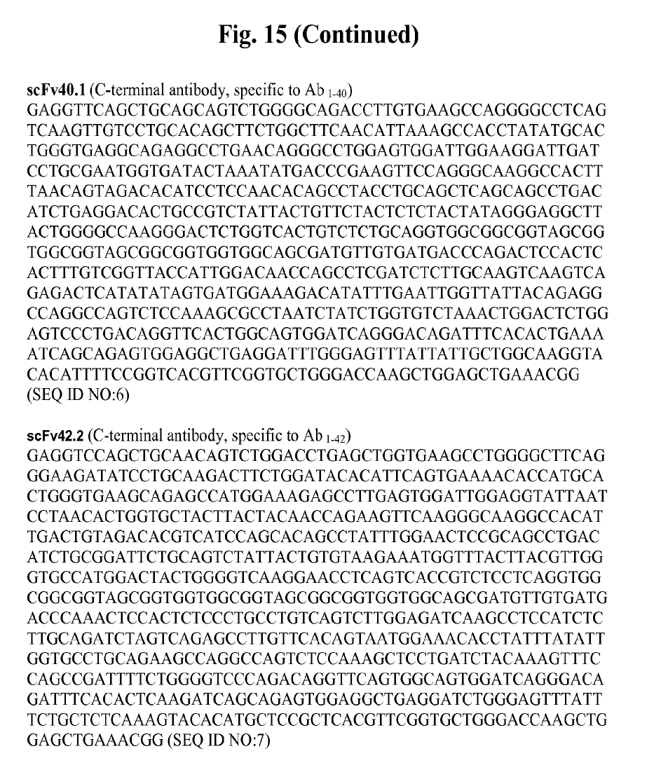

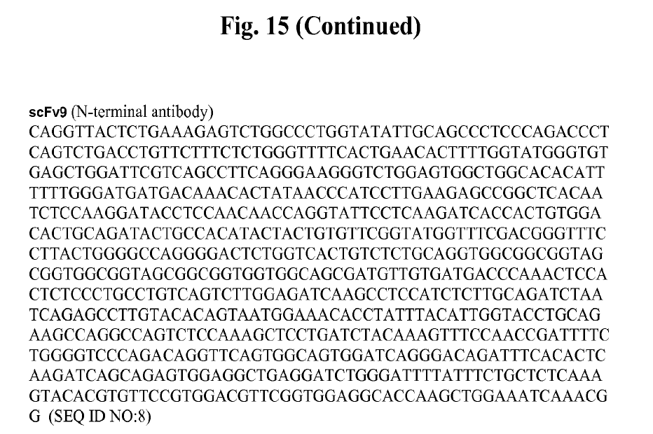

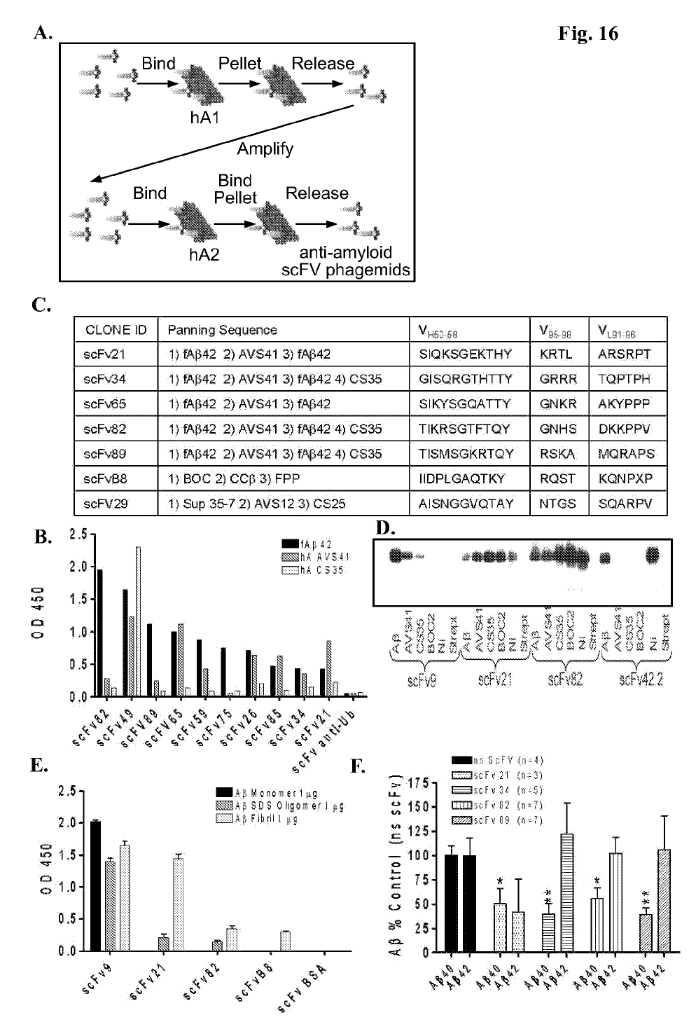

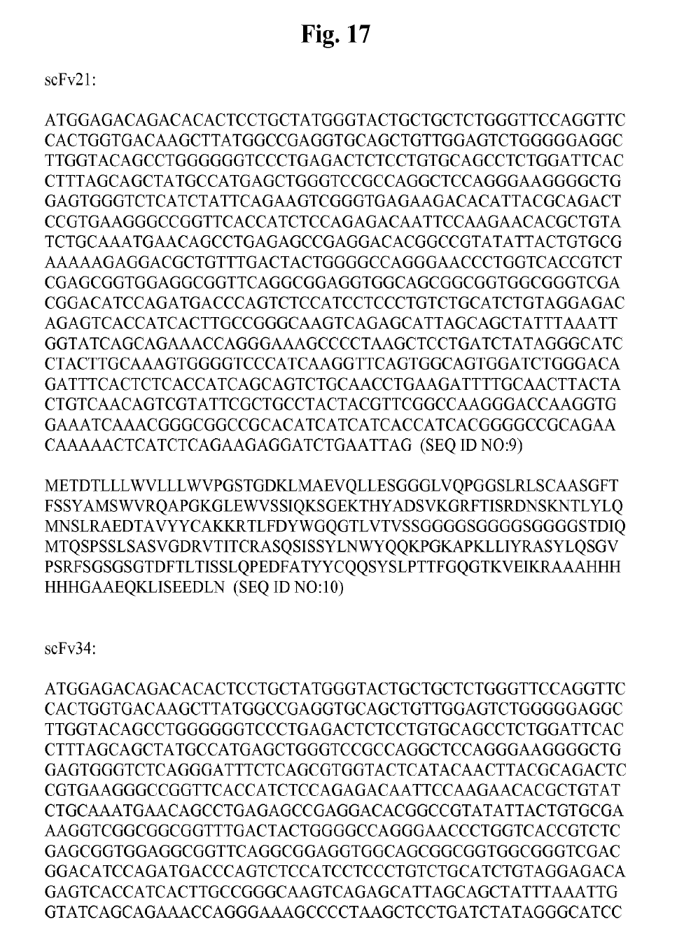

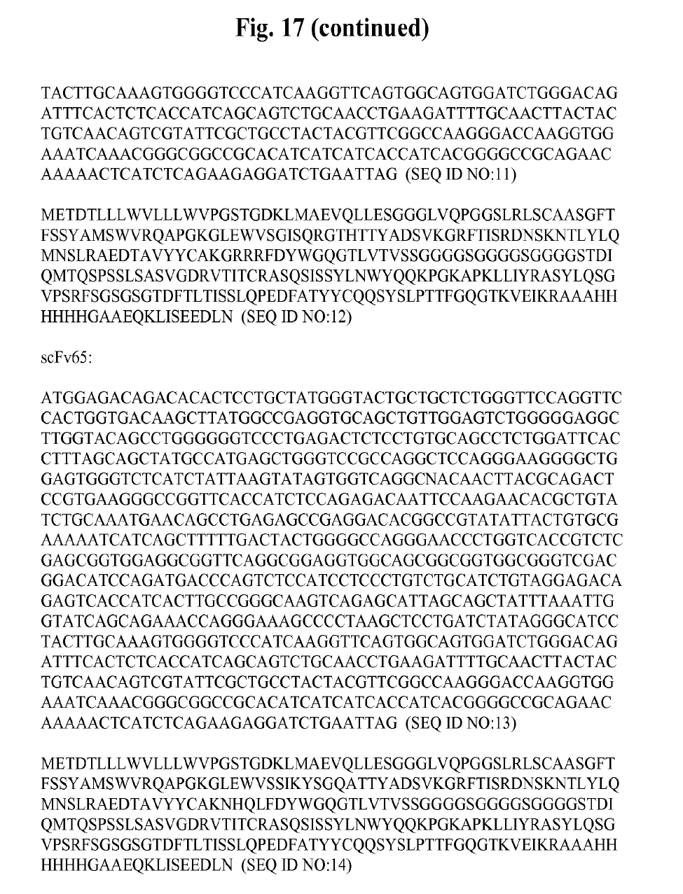

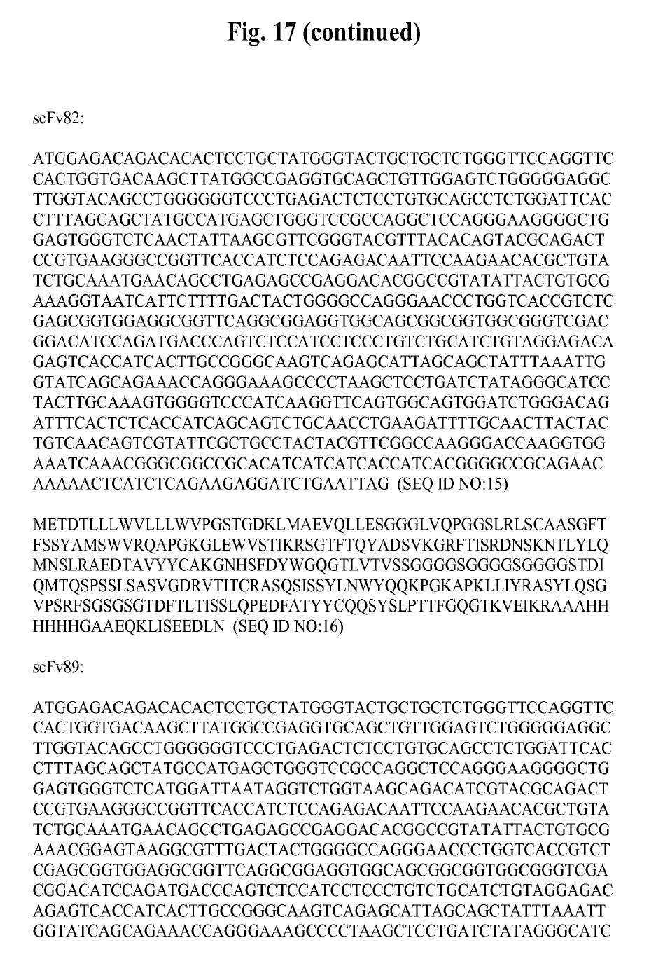

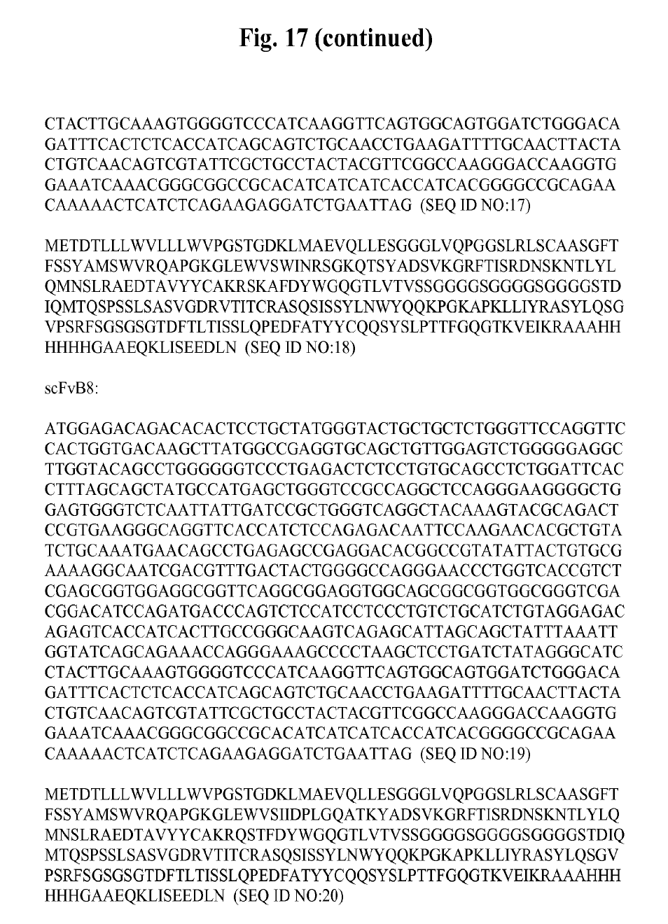

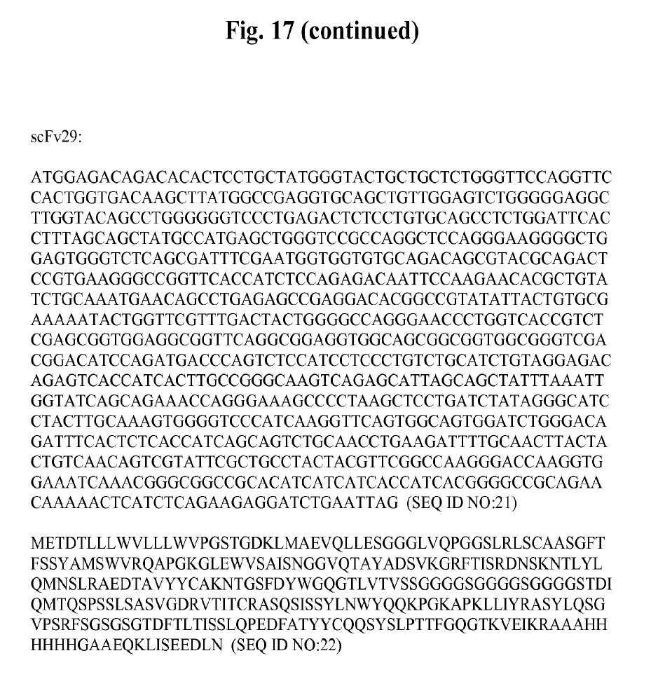

Anti-Aβ scFv reduce Aβ deposition in CRND8 mice. Initial studies were performed with the anti-pan Aβ scFv9 and the anti-Aβ42 specific scFv42.2. Control mice were injected with AAV1-hGFP. Following P0 injection, CRND8 mice were sacrificed at five months, and Aβ levels analyzed in the brain. Both anti-Aβ scFvs significantly attenuated Aβ40 and Aβ42 levels in SDS soluble (SDS) and SDS insoluble, FA-soluble (FA) extracts (FIG. 13). scFv9 and scFv42.2 reduced SDS and FA Aβ40 and Aβ42 respectively, and appeared to decrease immunoreactive Aβ loads as well (FIG. 13). A second more complete study was then conducted in CRND8 mice. Following P0 injection of AAV expressing scFV9, scFv42.2, scFv40.1, brain Aβ levels were analyzed in CRND8 mice at three months of age. In addition to a PBS injection control, an AAV1 expressing a non-specific scFv (scFv ns), which has no affinity to A13, was used as an additional control group. Aβ levels in scFv ns treated mice were not significantly different from control mice injected with PBS (FIG. 14B, C). In all scFv-treated mice, plaque loads were significantly decreased (FIG. 14A, B). Aβ40 and Aβ42 levels in SDS soluble fraction were also significantly reduced by all scFvs (FIG. 14C): scFv9 (25% and 20% reduction in Aβ40 and Aβ42, respectively); scFv40.1 (40% reduction in both Aβ40 and Aβ42); and scFV42.2 (30% and 20% reduction in Aβ40 and Aβ42, respectively). The largest effect was demonstrated by scFv40.1 possibly attributable to a higher expression level in the mouse brain. In this study, there was insufficient Aβ present in the FA fraction (in 3 month old mice) to make any reliable measurements. None of these studies showed any untoward side-effects. No increase in cerebral amyloid angiopathy or evidence for hemorrhage was seen.

A complex of scFv bound to Aβ was detected in the plasma of CRND8 mice by ELISA with an antibody specific to a free end of Aβ as capture and anti-myc-HRP as detection. For scFv9, mAb40.1 was used as capture. For scFv40.1 and scFv42.2, mAb9 was used as capture (FIG. 14D). The highest relative level of scFv-Aβ complex was detected for scFv40.1. This result suggests that scFv alone or in a complex with Aβ is cleared from the brain to the plasma.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A substantially pure antibody having binding affinity for an Aβ epitope, wherein said Aβ epitope is the epitope of scFv40.1 or scFv42.2.

2. The antibody of claim 1, wherein said antibody has less than 10⁴ mol⁻¹ binding affinity for Aβ1-38.

3. The antibody of claim 1, wherein said antibody has less than two percent cross reactivity with Aβ1-38.

4. The antibody of claim 1, wherein said antibody is monoclonal.

5. The antibody of claim 1, wherein said antibody comprises SEQ ID NO:2.

6. The antibody of claim 1, wherein said antibody comprises SEQ ID NO:3.

7. The antibody of claim 1, wherein said antibody is an scFv40.1 antibody.

8. The antibody of claim 1, wherein said antibody is an scFv42.2 antibody.

9. A method for inhibiting Aβ plaque formation in a mammal, said method comprising administering an antibody to said mammal, wherein said antibody has binding affinity for an Aβ epitope, wherein said Aβ epitope is the epitope of scFv40.1 or scFv42.2.

10. A nucleic acid construct comprising a nucleic acid sequence encoding the amino acid sequence set forth in SEQ ID NO:2 or 3.

11. The nucleic acid construct of claim 10, wherein said construct is an AAV vector.

12. A substantially pure antibody having binding affinity for an Aβ epitope, wherein said Aβ epitope is the epitope of scFv21, scFv34, scFv65, scFv82, scFv89, scFvB8, or scFv29.

13. The antibody of claim 12, wherein said antibody has less than 10⁴ mol⁻¹ binding affinity for Aβ1-38.

14. The antibody of claim 12, wherein said antibody has less than two percent cross reactivity with Aβ1-38.

15. The antibody of claim 12, wherein said antibody is monoclonal.

16. The antibody of claim 12, wherein said antibody comprises SEQ ID NO:10.

17. The antibody of claim 12, wherein said antibody comprises SEQ ID NO:12.

18. The antibody of claim 12, wherein said antibody comprises SEQ ID NO:14.

19. The antibody of claim 12, wherein said antibody comprises SEQ ID NO:16.

20. The antibody of claim 12, wherein said antibody comprises SEQ ID NO:18.

21. The antibody of claim 12, wherein said antibody comprises SEQ ID NO:20.

22. The antibody of claim 12, wherein said antibody comprises SEQ ID NO:22.

23. A method for inhibiting Aβ plaque formation in a mammal, said method comprising administering an antibody to said mammal, wherein said antibody has binding affinity for an Aβ epitope, wherein said Aβ epitope is the epitope of scFv21, scFv34, scFv65, scFv82, scFv89, scFvB8, or scFv29.

24. A nucleic acid construct comprising a nucleic acid sequence encoding the amino acid sequence set forth in SEQ ID NO:9, 11, 13, 15, 17, 19, or 21.

25. The nucleic acid construct of claim 24, wherein said construct is an AAV vector.


SEQUENCE LISTING (45 sequences)

SEQ ID NO: 1 - protein, 230 aa, Mus musculus
LVQPGGSLRLSCAASGFTFSSYAMSWVRQAPGKGLEWVSTIQRNGYYTRYADSVKGRFTISRDNSKNTLYLQMNSLRAEDTAVYYCAKKRFRFDYWGQGTLVTVSSGGGGSGGGGSGGGGSTDIQMTQSPSSLSASVGDRVTITCRASQSISSYLNWYQQKPGKAPKLLIYMASRLQSGVPSRFSGSGSGTDFTLTISSLQPEDFATYYCQQAWYLPSTFGQGTKVEIKR

SEQ ID NO: 2 - protein, 243 aa, Mus musculus
EVQLQQSGADLVKPGASVKLSCTASGFNIKATYMHWVRQRPEQGLEWIGRIDPANGDTKYDPKFQGKATLTVDTSSNTAYLQLSSLTSEDTAVYYCSTLYYREAYWGQGTLVTVSAGGGGSGGGSGGGGSDVVMTQTPLTLSVTIGQPASISCKSSQRLIYSDGKTYLNWLLQRPGQSPKRLIYLVSKLDSGVPDRFTGSGSGTDFTLKISRVEAEDLGVYYCWQGTHFPVTFGAGTKLELKR

SEQ ID NO: 3 - protein, 248 aa, Mus musculus
EVQLQQSGPELVKPGASGKISCKTSGYTFSENTMHWVKQSHGKSLEWIGGINPNTGATYYNQKFKGKATLTVDTSSSTAYLELRSLTSADSAVYYCVRNGLLTLGAMDYWGQGTSVTVSSGGGGSGGGGSGGGGSDVVMTQTPLSLPVSLGDQASISCRSSQSLVHSNGNTYLYWCLQKPGQSPKLLIYKVSSRFSGVPDRFSGSGSGTDFTLKISRVEAEDLGVYFCSQSTHAPLTFGAGTKLELKR

SEQ ID NO: 4 - protein, 246 aa, Mus musculus
  VARIANT 155: Xaa = Any Amino Acid
QVTLKESGPGILQPSQTLSLTCSFSGFSLNTFGMGVSWIRQPSGKGLEWLAHIFWDDDKHYNPSLKSRLTISKDTSNNQVFLKITTVDTADTATYYCVRYGFDGFPYWGQGTLVTVSAGGGGSGGGGSGGGGSDVVMTQTPLSLPVSLGDQASIXCRSNQSLVHSNGNTYLHWYLQKPGQSPKLLIYKVSNRFSGVPDRFSGSGSGTDFTLKISRVEAEDLGFYFYSQSTRVPWTFGGGTKLEIKR

SEQ ID NO: 5 - DNA, 690 bp, Mus musculus
ttggtacagcctggggggtccctgagactctcctgtgcagcctctggattcacctttagcagctatgccatgagctgggtccgccaggctccagggaaggggctggagtgggtctcaactattcagcggaatggttattatacacggtacgcagactccgtgaagggccggttcaccatctccagagacaattccaagaacacgctgtatctgcaaatgaacagcctgagagccgaggacacggccgtatattactgtgcgaaaaagaggtttcggtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatatggcatcccgtttgcaaagtggggtcccatcaaggttcagtggcagtggatctgggacagatttcactctcaccatcagcagtctgcaacctgaagattttgcaacttactactgccaacaggcttggtatcttccttctacgttcggccaagggaccaaggtggaaatcaaacgg

SEQ ID NO: 6 - DNA, 729 bp, Mus musculus
gaggttcagctgcagcagtctggggcagaccttgtgaagccaggggcctcagtcaagttgtcctgcacagcttctggcttcaacattaaagccacctatatgcactgggtgaggcagaggcctgaacagggcctggagtggattggaaggattgatcctgcgaatggtgatactaaatatgacccgaagttccagggcaaggccactttaacagtagacacatcctccaacacagcctacctgcagctcagcagcctgacatctgaggacactgccgtctattactgttctactctctactatagggaggcttactggggccaagggactctggtcactgtctctgcaggtggcggcggtagcggtggcggtagcggcggtggtggcagcgatgttgtgatgacccagactccactcactttgtcggttaccattggacaaccagcctcgatctcttgcaagtcaagtcagagactcatatatagtgatggaaagacatatttgaattggttattacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccggtcacgttcggtgctgggaccaagctggagctgaaacgg

SEQ ID NO: 7 - DNA, 744 bp, Mus musculus
gaggtccagctgcaacagtctggacctgagctggtgaagcctggggcttcagggaagatatcctgcaagacttctggatacacattcagtgaaaacaccatgcactgggtgaagcagagccatggaaagagccttgagtggattggaggtattaatcctaacactggtgctacttactacaaccagaagttcaagggcaaggccacattgactgtagacacgtcatccagcacagcctatttggaactccgcagcctgacatctgcggattctgcagtctattactgtgtaagaaatggtttacttacgttgggtgccatggactactggggtcaaggaacctcagtcaccgtctcctcaggtggcggcggtagcggtggtggcggtagcggcggtggtggcagcgatgttgtgatgacccaaactccactctccctgcctgtcagtcttggagatcaagcctccatctcttgcagatctagtcagagccttgttcacagtaatggaaacacctatttatattggtgcctgcagaagccaggccagtctccaaagctcctgatctacaaagtttccagccgattttctggggtcccagacaggttcagtggcagtggatcagggacagatttcacactcaagatcagcagagtggaggctgaggatctgggagtttatttctgctctcaaagtacacatgctccgctcacgttcggtgctgggaccaagctggagctgaaacgg

SEQ ID NO: 8 - DNA, 735 bp, Mus musculus
caggttactctgaaagagtctggccctggtatattgcagccctcccagaccctcagtctgacctgttctttctctgggttttcactgaacacttttggtatgggtgtgagctggattcgtcagccttcagggaagggtctggagtggctggcacacattttttgggatgatgacaaacactataacccatccttgaagagccggctcacaatctccaaggatacctccaacaaccaggtattcctcaagatcaccactgtggacactgcagatactgccacatactactgtgttcggtatggtttcgacgggtttccttactggggccaggggactctggtcactgtctctgcaggtggcggcggtagcggtggcggtagcggcggtggtggcagcgatgttgtgatgacccaaactccactctccctgcctgtcagtcttggagatcaagcctccatctcttgcagatctaatcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggattttatttctgctctcaaagtacacgtgttccgtggacgttcggtggaggcaccaagctggaaatcaaacgg

SEQ ID NO: 9 - DNA, 867 bp, Mus musculus
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattcagaagtcgggtgagaagacacattacgcagactccgtgaagggccggttcaccatctccagagacaattccaagaacacgctgtatctgcaaatgaacagcctgagagccgaggacacggccgtatattactgtgcgaaaaagaggacgctgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 10 - protein, 288 aa, Mus musculus
METDTLLLWVLLLWVPGSTGDKLMAEVQLLESGGGLVQPGGSLRLSCAASGFTFSSYAMSWVRQAPGKGLEWVSSIQKSGEKTHYADSVKGRFTISRDNSKNTLYLQMNSLRAEDTAVYYCAKKRTLFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 11 - DNA, 867 bp, Mus musculus
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagggatttctcagcgtggtactcatacaacttacgcagactccgtgaagggccggttcaccatctccagagacaattccaagaacacgctgtatctgcaaatgaacagcctgagagccgaggacacggccgtatattactgtgcgaaaggtcggcggcggtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 12 - protein, 288 aa, Mus musculus
METDTLLLWVLLLWVPGSTGDKLMAEVQLLESGGGLVQPGGSLRLSCAASGFTFSSYAMSWVRQAPGKGLEWVSGISQRGTHTTYADSVKGRFTISRDNSKNTLYLQMNSLRAEDTAVYYCAKGRRRFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 13 - DNA, 867 bp, Mus musculus
  misc_feature 246: n = A,T,C or G
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattaagtatagtggtcaggcnacaacttacgcagactccgtgaagggccggttcaccatctccagagacaattccaagaacacgctgtatctgcaaatgaacagcctgagagccgaggacacggccgtatattactgtgcgaaaaatcatcagctttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 14 - protein, 288 aa, Mus musculus
METDTLLLWVLLLWVPGSTGDKLMAEVQLLESGGGLVQPGGSLRLSCAASGFTFSSYAMSWVRQAPGKGLEWVSSIKYSGQATTYADSVKGRFTISRDNSKNTLYLQMNSLRAEDTAVYYCAKNHQLFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 15 - DNA, 867 bp, Mus musculus
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaactattaagcgttcgggtacgtttacacagtacgcagactccgtgaagggccggttcaccatctccagagacaattccaagaacacgctgtatctgcaaatgaacagcctgagagccgaggacacggccgtatattactgtgcgaaaggtaatcattcttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 16 - protein, 288 aa, Mus musculus
METDTLLLWVLLLWVPGSTGDKLMAEVQLLESGGGLVQPGGSLRLSCAASGFTFSSYAMSWVRQAPGKGLEWVSTIKRSGTFTQYADSVKGRFTISRDNSKNTLYLQMNSLRAEDTAVYYCAKGNHSFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 17 - DNA, 867 bp, Mus musculus
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatggattaataggtctggtaagcagacatcgtacgcagactccgtgaagggccggttcaccatctccagagacaattccaagaacacgctgtatctgcaaatgaacagcctgagagccgaggacacggccgtatattactgtgcgaaacggagtaaggcgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 18 - protein, 288 aa, Mus musculus
METDTLLLWVLLLWVPGSTGDKLMAEVQLLESGGGLVQPGGSLRLSCAASGFTFSSYAMSWVRQAPGKGLEWVSWINRSGKQTSYADSVKGRFTISRDNSKNTLYLQMNSLRAEDTAVYYCAKRSKAFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 19 - DNA, 867 bp, Mus musculus
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaattattgatccgctgggtcaggctacaaagtacgcagactccgtgaagggcaggttcaccatctccagagacaattccaagaacacgctgtatctgcaaatgaacagcctgagagccgaggacacggccgtatattactgtgcgaaaaggcaatcgacgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 20 - protein, 288 aa, Mus musculus
METDTLLLWVLLLWVPGSTGDKLMAEVQLLESGGGLVQPGGSLRLSCAASGFTFSSYAMSWVRQAPGKGLEWVSIIDPLGQATKYADSVKGRFTISRDNSKNTLYLQMNSLRAEDTAVYYCAKRQSTFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 21 - DNA, 867 bp, Mus musculus
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagcgatttcgaatggtggtgtgcagacagcgtacgcagactccgtgaagggccggttcaccatctccagagacaattccaagaacacgctgtatctgcaaatgaacagcctgagagccgaggacacggccgtatattactgtgcgaaaaatactggttcgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 22 - protein, 288 aa, Mus musculus
METDTLLLWVLLLWVPGSTGDKLMAEVQLLESGGGLVQPGGSLRLSCAASGFTFSSYAMSWVRQAPGKGLEWVSAISNGGVQTAYADSVKGRFTISRDNSKNTLYLQMNSLRAEDTAVYYCAKNTGSFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 23 - protein, 15 aa, Artificial Sequence
  Synthetically generated polypeptide
AALALPAVLLALLAP

SEQ ID NO: 24 - protein, 17 aa, Artificial Sequence
  Synthetically generated polypeptide
KGEGAAVLLPVLLAAPG

SEQ ID NO: 25 - protein, 11 aa, Mus musculus
SIQKSGEKTHY

SEQ ID NO: 26 - protein, 11 aa, Mus musculus
GISQRGTHTTY

SEQ ID NO: 27 - protein, 11 aa, Mus musculus
SIKYSGQATTY

SEQ ID NO: 28 - protein, 11 aa, Mus musculus
TIKRSGTFTQY

SEQ ID NO: 29 - protein, 11 aa, Mus musculus
TISMSGKRTQY

SEQ ID NO: 30 - protein, 11 aa, Mus musculus
IIDPLGAQTKY

SEQ ID NO: 31 - protein, 11 aa, Mus musculus
AISNGGVQTAY

SEQ ID NO: 32 - protein, 4 aa, Mus musculus
KRTL

SEQ ID NO: 33 - protein, 4 aa, Mus musculus
GRRR

SEQ ID NO: 34 - protein, 4 aa, Mus musculus
GNKR

SEQ ID NO: 35 - protein, 4 aa, Mus musculus
GNHS

SEQ ID NO: 36 - protein, 4 aa, Mus musculus
RSKA

SEQ ID NO: 37 - protein, 4 aa, Mus musculus
RQST

SEQ ID NO: 38 - protein, 4 aa, Mus musculus
NTGS

SEQ ID NO: 39 - protein, 6 aa, Mus musculus
ARSRPT

SEQ ID NO: 40 - protein, 6 aa, Mus musculus
TQPTPH

SEQ ID NO: 41 - protein, 6 aa, Mus musculus
AKYPPP

SEQ ID NO: 42 - protein, 6 aa, Mus musculus
DKKPPV

SEQ ID NO: 43 - protein, 6 aa, Mus musculus
MQRAPS

SEQ ID NO: 44 - protein, 6 aa, Mus musculus
  VARIANT 5: Xaa = Any Amino Acid
KQNPXP

SEQ ID NO: 45 - protein, 6 aa, Mus musculus
SQARPV